(19) United States
(12) Patent Application Publication
(10) Pub. No.: US 2025/0195290 A1
(43) Pub. Date: Jun. 19, 2025

(54) ABSORBENT ARTICLES WITH FRANGIBLE PATHWAYS COMPRISING LINES OF WEAKNESS WITH FREE FIBER ENDS

(71) Applicant: The Procter & Gamble Company, Cincinnati, OH (US)

(72) Inventors: Jeromy Thomas RAYCHECK, South Lebanon, OH (US); Keith Richard WILLHAUS, Cincinnati, OH (US); Paul Thomas WEISMAN, Cincinnati, OH (US); Masaharu NISHIKAWA, Cincinnati, OH (US)

(21) Appl. No.: 18/978,059

(22) Filed: Dec. 12, 2024

Related U.S. Application Data
(60) Provisional application No. 63/610,481, filed on Dec. 15, 2023.

(51) Int. Cl. A61F 13/49

(52) U.S. Cl. CPC A61F 13/49011; A61F 2013/49087

(57) ABSTRACT

The present disclosure relates to absorbent articles having front and/or back waist regions including one or more frangible pathways comprising lines of weakness with free fiber ends.

### CROSS REFERENCE TO RELATED APPLICATION

This application claims the benefit of U.S. Provisional Application No. 63/610,481, filed Dec. 15, 2023, which is incorporated by reference herein in its entirety.

### FIELD

The present disclosure relates to absorbent articles, and more particularly, to absorbent articles having front and/or back waist regions including one or more frangible pathways comprising lines of weakness with free fiber ends.

### BACKGROUND

Some absorbent articles have components that include elastomeric laminates. Such elastomeric laminates may include an elastic material bonded to one or more nonwovens. The elastic material may include an elastic film and/or elastic strands. In some laminates, a plurality of elastic strands are joined to a nonwoven while the plurality of strands are in a stretched condition so that when the elastic strands relax, the nonwoven gathers, and in turn, forms corrugations and rugosities. The resulting elastomeric laminate is stretchable to the extent that the corrugations allow the elastic strands to elongate.

Absorbent articles in the form of diaper pants may also be configured with an absorbent chassis connected with front and back elastic belts, wherein opposing end regions of the front and back belts are connected with each other at side seams. In some instances, the elasticity of the front and back belts is removed in regions where the chassis connects with the belts. Thus, in some converting configurations adapted to assemble such diaper pants, stretched elastic strands are glued between two continuous nonwoven webs to form an elastic laminate. Regions of the elastic strands may then be intermittently deactivated along the length of the elastic laminate by cutting the elastic strands in areas to be connected with the chassis, sometimes referred to as tummy elastic cutting.

Some caregivers of older incontinent babies or toddlers may prefer a closed, pant-style disposable absorbent article to enable application to, and removal from, a child while the child is in a standing position. One disadvantage of this product form is that the removal and disposal of feces-containing products may be unhygienic and inconvenient. For example, pulling the product down could cause feces to smear down the legs of a wearer. In other examples, a caregiver may tear open the bonded sides using force. In turn, the force used can lead to a rapid release of energy from the diaper, causing the caregiver to lose control of the product and allowing feces to spill out. In contrast, removal and disposal of traditional open or taped diaper forms with fasteners may be readily accomplished while the child is laying on their back. In this case, the fasteners are opened, the diaper is removed from under the child, rolled into a roughly cylindrical shape, and then the fasteners are secured around the rolled, soiled diaper, closing the leg openings for hygienic disposal.

In order to avoid having to remove soiled diaper pants from a wearer by sliding the soiled diaper pant down the wearer's legs or tearing bonded side seams, some diaper pants may be configured with tear lines in the front belt or back belt. Such tear lines may include perforations that allow a caregiver to more easily separate the belt along the perforation lines. Once the belt is separated, the diaper pant can be more easily removed from the wearer without having to slide the diaper pant down the wearer's legs, in a similar manner as a traditional open taped diaper form. However, existing configurations of tear lines and associated perforations may have certain drawbacks.

For example, in certain situations, some perforations may be highly visible to the caregiver and/or wearer. Such visibility may draw unwanted attention from a curious wearer of a diaper pant, such as a toddler. In turn, the wearer may be inclined to insert fingers into the perforations and begin to tear the belt. In some circumstances, perforations may tend to open a certain extent when the belt is stretched while by being worn. Such visible and/or opened perforations may have additional unintended consequences. For example, opened perforations may unintentionally provide an area where a wearer may relatively easily insert fingers to tear the belt while being worn. In addition, highly visible and/or opened perforations may give the impression to the caregiver that the belt is weak and may break during normal use.

In other situations, knives used in cutting operations to create perforations in belts may create perforations comprising apertures with perimeter edges defined by belt material that has been melted and/or fused together due to relatively high knife and anvil pressures when the belt material is cut. In turn, the melted and/or fused material of the perforation edges may be exposed to the caregiver and/or wearer once the belt has been torn during removal from a wearer. Such melted and/or fused material may feel sharp and/or rough, unintentionally providing an unwanted and/or negative tactile signal to the caregiver and/or wearer, resulting in a negative consumer experience.

Consequently, it would be beneficial to create pant-style articles that provide the caregiver the ability to remove and dispose soiled products in a similar manner to traditional open diaper forms. In addition, it would be beneficial to provide diaper pants with frangible pathways configured with lines of weakness that resist opening while the diaper pant is being worn. Further, it would be beneficial to provide diaper pants with frangible pathways configured with lines of weakness that may be relatively less visually detectable and/or mitigate situations wherein a torn belt edge may feel rough or sharp.

### SUMMARY

In one form, an absorbent article comprises: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first free fiber end density, and wherein the torn land regions comprise a second free fiber end density; and wherein the second free fiber end density is greater than the first free fiber end density.

In another form, an absorbent article comprises: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first free fiber end density, and wherein the torn land regions comprise a second free fiber end density; and wherein a ratio of the first free fiber end density to the second free fiber end density is less than 0.5.

In yet another form, an absorbent article comprises: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first length and a first free fiber end density, and wherein the torn land regions comprise a second length and a second free fiber end density; and wherein the second free fiber end density times the second length of the torn land region is greater than the first free fiber end density times the first length of the first cut edge.

In still another form, an absorbent article comprises: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side scam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first free fiber end density, and wherein the torn land regions comprise a second free fiber end density; and wherein the second free fiber end density is from about 2 free fiber ends/mm to about 10000 free fiber ends/mm.

In still another form, an absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side scam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; and a plurality of fibers of the fibrous substrate extending at least partially across the cut line between the first cut edge and the second cut edge; and wherein the first belt is separable along the frangible pathway with a tear line propagating through the cut lines and the land regions to define a first edge of a first belt zone and a second edge of a second belt zone.

In still another form, an absorbent article comprises: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side scam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions; and wherein the first edge comprises a Loose Fiber Ends Value of about 35 to about 65.

In still another form, an absorbent article comprises: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions; and wherein the cut line comprises a Cut Line Occlusion of about 15% to about 80%.

### BRIEF DESCRIPTION OF THE DRAWINGS

FIG. 1 shows a perspective view of a diaper pant in a pre-fastened configuration.

FIG. 1A shows a perspective view of a diaper pant with a continuous outer cover in a pre-fastened configuration.

FIG. 2A shows a plan view of a diaper pant with the portion of the diaper that faces away from a wearer oriented toward the viewer.

FIG. 2B shows a plan view of a diaper pant with the portion of the diaper that faces toward a wearer oriented toward the viewer.

FIG. 2C shows a plan view of a diaper pant with the portion of the diaper that faces away from a wearer oriented toward the viewer, illustrating first and second belt size and shape features.

FIG. 2D shows a plan view of a diaper pant with the portion of the diaper that faces away from a wearer oriented toward the viewer, illustrating first and second belt size and shape features.

FIG. 2E shows a plan view of a diaper pant with the portion of the diaper that faces away from a wearer oriented toward the viewer, illustrating first and second belt size and shape features.

FIG. 2F shows a plan view of a diaper pant with a continuous outer cover with the portion of the diaper that faces away from a wearer oriented toward the viewer.

FIG. 3 is a cross-sectional view of the diaper pant of FIG. 2A taken along line 3-3 showing first and second elastic belts provided with panel layers.

FIG. 3A is a cross-sectional detailed view of a first belt provided with panel layers wherein one panel layer is folded over another panel layer.

FIG. 3A1 is a cross-sectional detailed view of another example configuration wherein the first belt is provided with panel layers wherein one panel layer is folded over another panel layer.

FIG. 3A2 is a cross-sectional detailed view of another example configuration wherein the first belt is provided with panel layers wherein one panel layer is folded over another panel layer.

FIG. 3B is a cross-sectional detailed view of a second belt provided with panel layers wherein one panel layer is folded over another panel layer.

FIG. 3C is a cross-sectional view of the diaper pant of FIG. 2F taken along line 3C-3C showing first and second elastic belts provided with panel layers and a continuous outer cover.

FIG. 4A is perspective view of a diaper pant including frangible pathways in a front belt and adjacent an absorbent chassis.

FIG. 4B is a perspective view of the diaper pant of FIG. 4A showing the front belt having been torn along one of the frangible pathways.

FIG. 4C is a perspective view of the diaper pant of FIG. 4A showing the front belt having been torn along two frangible pathways.

FIG. 5A shows the diaper pant of FIG. 4C being rolled up onto itself in a longitudinal direction.

FIG. 5B shows the diaper pant of FIG. 5A with fastener components connected with the backsheet of the chassis to maintain the diaper pant in a disposal configuration.

FIG. 6A is a perspective view of a diaper pant with frangible pathways.

FIG. 6B is a front plan view of the diaper pant of FIG. 6A.

FIG. 6C shows a front plan view of the diaper pant of FIG. 6B as a first frangible pathway is being torn.

FIG. 6D shows a front plan view of the diaper pant of FIG. 6C after the first frangible pathway has been completely torn.

FIG. 6E shows a front plan view of the diaper pant of FIG. 6D as a second frangible pathway is being torn.

FIG. 6F shows a front plan view of the diaper pant of FIG. 6E after the second frangible pathway has been completely torn.

FIG. 7A is a front plan view of another configuration of a diaper pant with frangible pathways having a distal terminus and a proximal terminus positioned on side seams.

FIG. 7B is a front plan view of another configuration of a diaper pant with frangible pathways having an accessibility opening positioned longitudinally between the fastener component and an inner edge of the first belt.

FIG. 7C shows a front plan view of a diaper pant with another configuration of frangible pathways.

FIG. 7D shows a front plan view of a diaper pant with another configuration of frangible pathways.

FIG. 8A is a detailed view of a fastener component configuration of FIG. 6A.

FIG. 8AA1 is a cross-sectional view of the fastener component of FIG. 8A taken along line 8AA-8AA.

FIG. 8AA2 is a cross-sectional view of the fastener component of FIG. 8A taken along line 8AA-8AA, wherein the fastener component is integrally formed from belt components.

FIG. 9A is a detailed view of another fastener component configuration of FIG. 7A.

FIG. 9B is a detailed view of another fastener component configuration.

FIG. 10A is a detailed sketch of lines of weakness of a frangible pathway in a fibrous substrate.

FIG. 10B is a detailed sketch of the frangible pathway of FIG. 10A after having been separated along a tear line.

FIG. 11A is a microscope photograph of a line of weakness made with a first cutting pressure and showing a relatively few number of remainder fibers extending between first and second cut edges of a cut line.

FIG. 11B is a microscope photograph of a line of weakness made with a second cutting pressure that is lower than the first cutting pressure and showing a relatively large number of remainder fibers extending between first and second cut edges of a cut line.

### DETAILED DESCRIPTION

### Definitions

The following term explanations may be useful in understanding the present disclosure: “Absorbent article” refers to devices, which absorb and contain body exudates and, more specifically, refers to devices, which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, menstrual pads and the like.

“Body-facing” and “garment-facing” refer respectively to the relative location of an element or a surface of an element or group of elements. “Body-facing” implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

The terms “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. Elastomeric materials may include elastomeric films, scrims, nonwovens, ribbons, strands and other sheet-like structures.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “distal” is used to describe a position situated away from a center of a body or from a point of attachment, and the term “proximal” is used to describe a position situated nearer to a center of a body or a point of attachment.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. In some configurations, a nonwoven may comprise a polyolefin based nonwoven, including but not limited to nonwovens having polypropylene fibers and/or polyethylene fibers and/or bicomponent fibers comprising a polyolefin. Nonlimiting examples of suitable fibers include spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers as known in the art, and workable combinations thereof. Nonwovens do not have a woven or knitted filament pattern. It is to be appreciated that nonwovens having various basis weights can be used in accordance with the methods herein. For example, some nonwovens may have a basis weight of at least about 8 gsm, 12 gsm, 16 gsm, 20 gsm, 25 gsm, 25 gsm, 40 gsm, or 65 gsm. Some nonwovens may have basis weight of about 8 gsm to about 65 gsm, specifically reciting all 1 gsm increments within the above-recited ranges and all ranges formed therein or thereby. Some nonwovens may be configured with meltblown layers that may provide barrier properties. For example, some nonwoven fabric webs may comprise spunbond, meltblown, spunbond (“SMS”) webs comprising outer layers of spunbond thermoplastics (e.g., polyolefins) and an interior layer of meltblown thermoplastics. Such SMS nonwoven fabric webs may comprise spunbond layers which are durable and an internal meltblown layer which is porous but which may inhibit fast strikethrough of fluids, such as bodily fluids, for example, or the penetration of bacteria through the fabric webs. In some configurations, the meltblown layer may have a fiber size and a porosity that assures breathability of the nonwoven fabric web while at the same time inhibiting the strikethrough of fluids. In some configurations, a nonwoven component layer may comprise fine fibers (“N-fibers”) with an average diameter of less than 1 micron (an “N-fiber layer”) that may be added to, or otherwise incorporated with, other nonwoven component layers to form a nonwoven web of material. For example, the N-fiber layer may be used to produce a SNS nonwoven web or a SMNS nonwoven web. As such, nonwoven web materials may be an SMS material, comprising a spunbonded, a melt-blown and a further spunbonded stratum or layer or any other combination of spunbonded and melt-blown layers, such as a SMMS or SSMMS. Some examples may include one or more layers of fibers with diameters below 1 micron (nanofibers and nanofiber layers); examples of these rise in combinations of SMS, SMNS, SSMNS or SMNMS nonwoven webs (where “N” designates a nanofiber layer). Various examples of nonwovens, fiber compositions, formations of fibers, and nonwovens and related methods are described in U.S. Pat. Nos. 6,645,569; 6,863,933; 7,112,621; and 8,728,051, which are incorporated by reference herein.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

“Pre-strain” refers to the strain imposed on an elastic or elastomeric material prior to combining it with another element of the elastomeric laminate or the absorbent article. Pre-strain is determined by the following equation Pre-strain=((extended length of the elastic-relaxed length of the elastic)/relaxed length of the elastic)*100.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, back waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1, 2012/0061016 A1, 2012/0061015 A1; 2013/0255861 A1; 2013/0255862 A1; 2013/0255863 A1; 2013/0255864 A1; and 2013/0255865 A1, all of which are incorporated by reference herein.

“Closed-form” means opposing waist regions are joined, as packaged, either permanently or refastenably to form a continuous waist opening and leg openings.

“Open-form” means opposing waist regions are not initially joined to form a continuous waist opening and leg openings but comprise a closure means such as a fastening system to join the waist regions to form the waist and leg openings before or during application to a wearer of the article.

The present disclosure relates to absorbent articles having front and/or back waist regions including one or more frangible pathways. As discussed in more detail below, an absorbent article may comprising: a first belt and a second belt, wherein laterally opposing end portions of the second belt are connected with laterally opposing end portions of the first belt at a first side seam and a second side seam to form a waist opening. The absorbent article may further comprise a chassis comprising a topsheet, a backsheet, and an absorbent core positioned between the topsheet and the backsheet, and longitudinally opposing end regions of the chassis are connected with the first belt and the second belt. The first belt may further comprise at least one layer of a fibrous substrate and a frangible pathway. The frangible pathway may comprise lines of weakness comprising cut lines extending through the fibrous substrate, each line of weakness being separated from another line of weakness other by land regions. Each cut line comprises a first cut edge separated from a second cut edge, and each land region comprises an uncut region of the fibrous substrate, such as a nonwoven for example. A plurality of fibers of the fibrous substrate extend at least partially across the cut line between the first cut edge and the second cut edge.

As discussed in more detail below, the first belt is separable along the frangible pathway with a tear line propagating through the cut lines and the land regions to define a first edge of a first belt zone and a second edge of a second belt zone, wherein the first edge of the first belt zone comprises first cut edges separated from each other by torn land regions. The first cut edges comprise a first free fiber end density, and the torn land regions comprise a second free fiber end density. The first free fiber density and/or the second free fiber end density may be greater than zero. In some configurations, the second free fiber end density is greater than the first free fiber end density. In some configurations, a ratio of the first free fiber end density to the second free fiber end density is less than 0.5. In some configurations, the first cut edges may comprise a first length and a first free fiber end density, and the torn land regions may comprise a second length and a second free fiber end density, and the second free fiber end density times the second length of the torn land region may be greater than the first free fiber end density times the first length of the first cut edge. In some configurations, the first free fiber end density is about 5% to about 60% of a sum of the first free fiber end density and the second free fiber end density. In some configurations, the second free fiber end density is from about 2 free fiber ends/mm to about 10000 free fiber ends/mm.

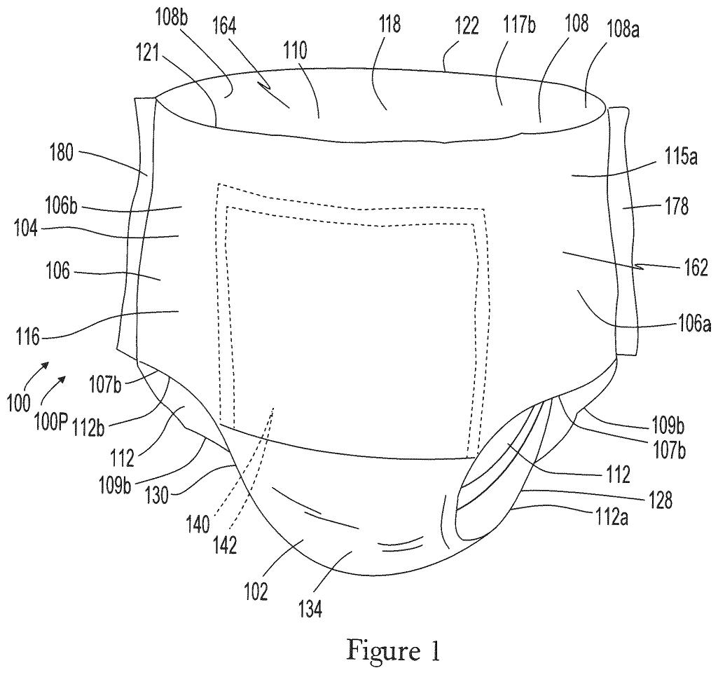

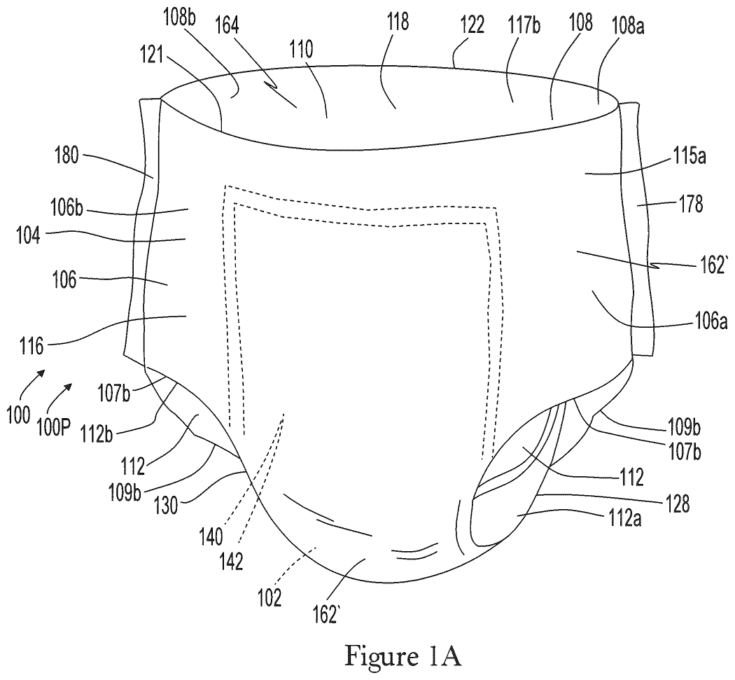

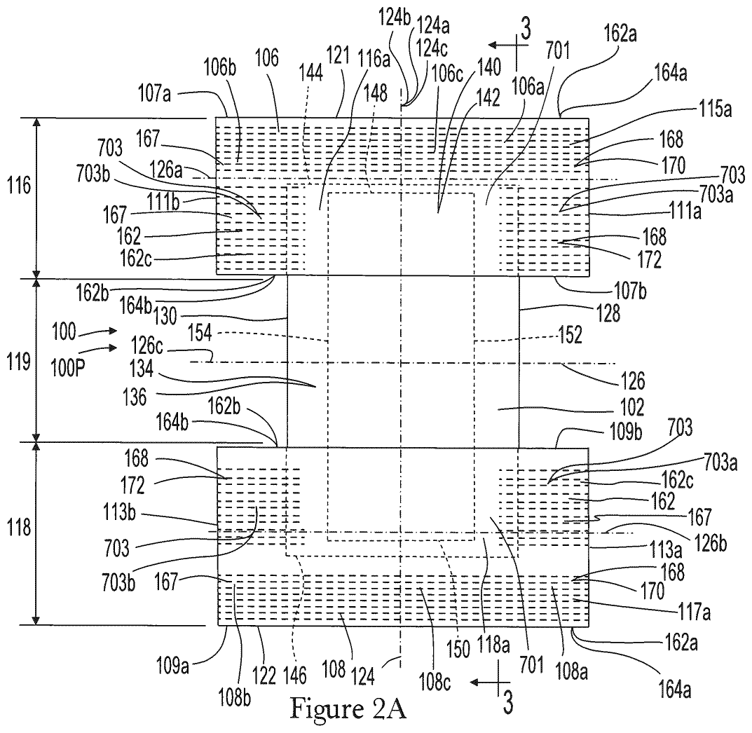

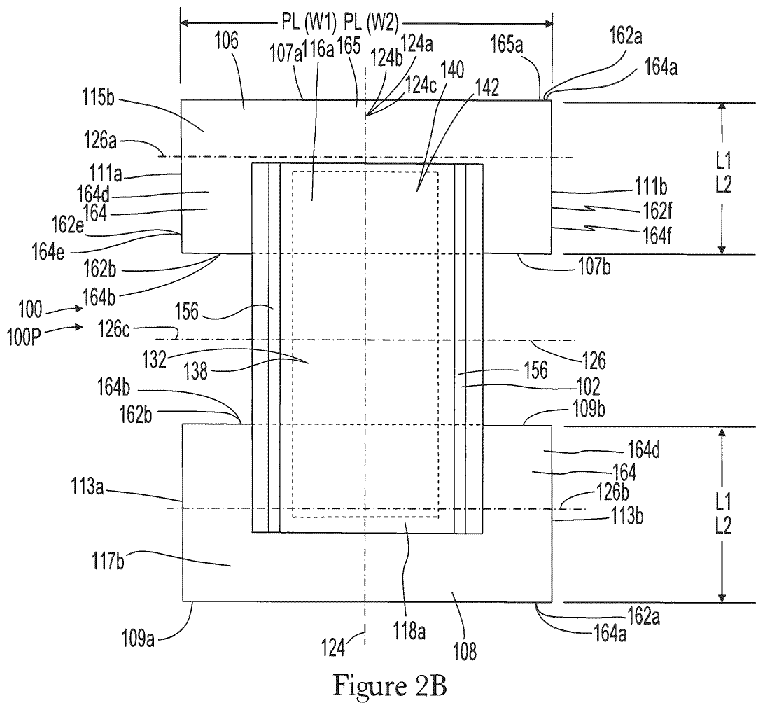

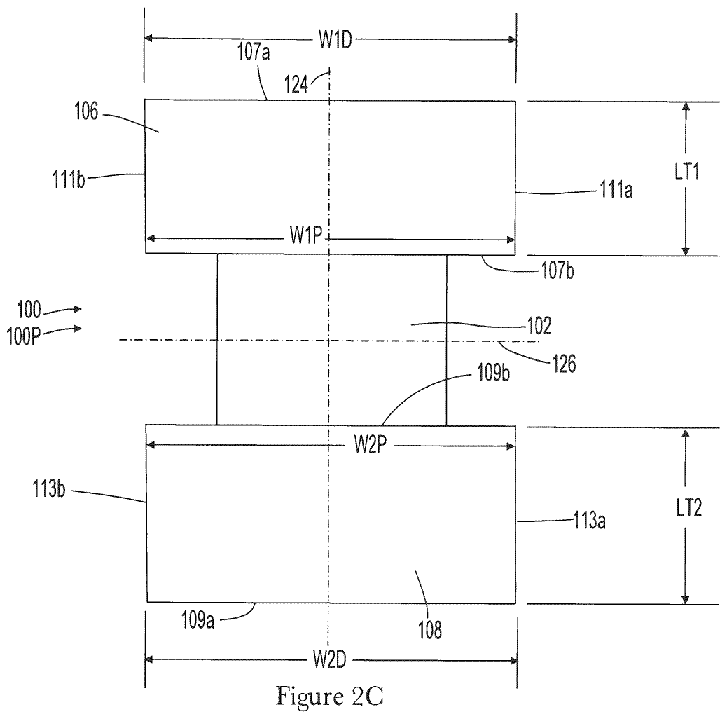

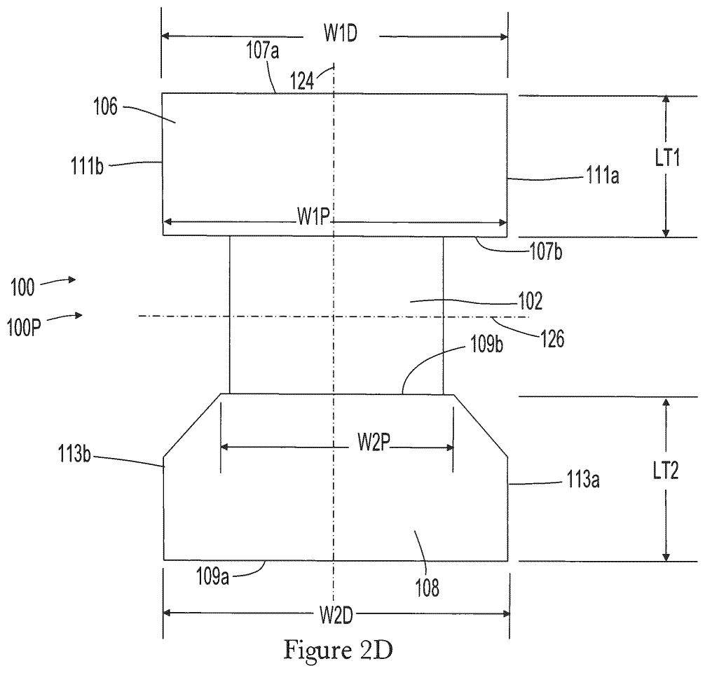

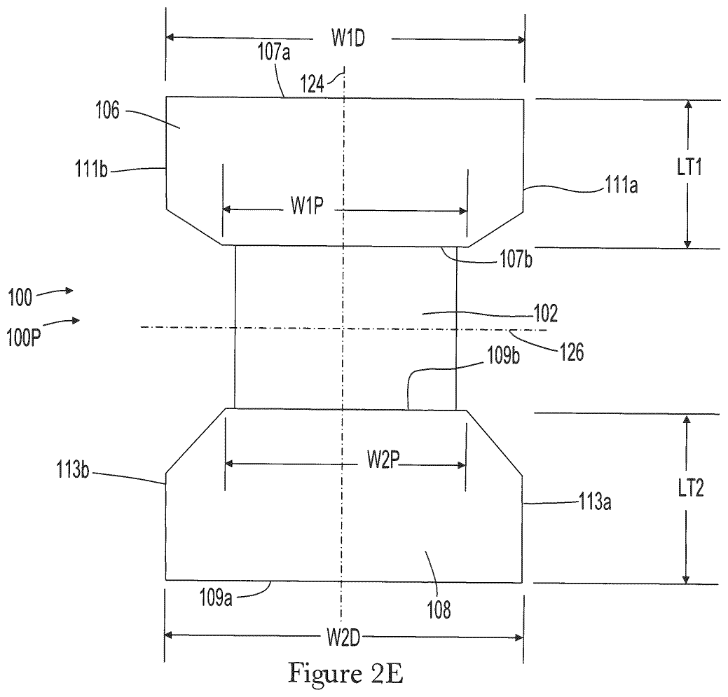

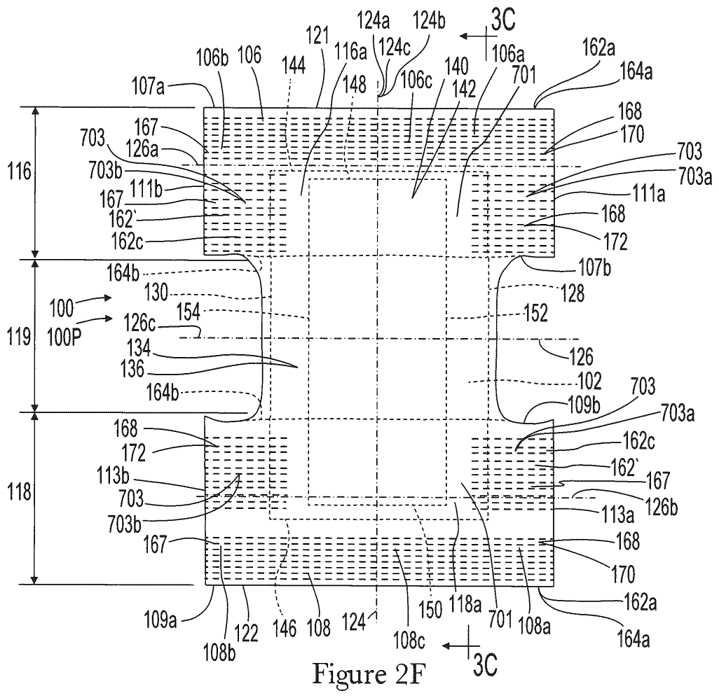

FIGS. 1-2B show an example of an absorbent article 100 in the form of a diaper pant 100P that may include components that may be constructed in accordance with the configurations disclosed herein. In particular, FIG. 1 shows a perspective views of a diaper pant 100P in a pre-fastened configuration. FIG. 2A shows a plan view of the diaper pant 100P with the portion of the diaper that faces away from a wearer oriented toward the viewer, and FIG. 2B shows a plan view of the diaper pant 100P with the portion of the diaper that faces toward a wearer oriented toward the viewer. The diaper pant 100P includes a chassis 102 and a ring-like elastic belt 104. As discussed below in more detail, a first elastic belt 106 and a second elastic belt 108 are bonded together to form the ring-like elastic belt 104.

With continued reference to FIGS. 1-2B, the diaper pant 100P and the chassis 102 each include a first waist region 116, a second waist region 118, and a crotch region 119 disposed intermediate the first and second waist regions. It may also be described that the chassis 102 includes a first end region 116a, a second end region 118a, and a crotch region 119 disposed intermediate the first and second end regions 116a, 118a. The first waist region 116 may be configured as a front waist region, and the second waist region 118 may be configured as back waist region. The diaper 100P may also include a laterally extending front waist edge 121 in the front waist region 116 and a longitudinally opposing and laterally extending back waist edge 122 in the back waist region 118. To provide a frame of reference for the present discussion, the diaper 100P and chassis 102 of FIGS. 2A and 2B are shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a second longitudinal or left side edge 130 of the chassis 102. As previously mentioned, the longitudinal axis 124 extends perpendicularly through the front waist edge 121 and the back waist edge 122, and the lateral axis 126 extends perpendicularly to the longitudinal axis 124. When the diaper pant 100P is worn, the longitudinal direction may extend from the wearer's front waist, through the crotch, to the wearer's back waist. To provide a further frame of reference for the present discussion, the diapers 100P of FIGS. 2A, 2B, and 18B are shown wherein: the first clastic belt 106 comprises a longitudinal centerline 124a and lateral centerline 126a; the second elastic belt 108 comprises a longitudinal centerline 124b and lateral centerline 126b; and the chassis 102 comprises a longitudinal centerline 124c and lateral centerline 126c. The longitudinal centerlines 124a, 124b, 124c are perpendicular to the lateral center lines 126a, 126b, 126c.

As shown in FIGS. 1-2B, the diaper pant 100P may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100P may also include other features, such as leg elastics and/or leg cuffs to enhance the fit around the legs of the wearer.

As shown in FIG. 2A, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 2A, the laterally extending end edges 144 and 146 may be located longitudinally inward from the laterally extending front waist edge 121 in the front waist region 116 and the laterally extending back waist edge 122 in the back waist region 118. In some configurations, the laterally extending end edges 144 and 146 may be coterminous with or located longitudinally outward from the laterally extending front waist edge 121 in the front waist region 116 and the laterally extending back waist edge 122 in the back waist region 118. When the diaper pant 100P is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

As previously mentioned, the diaper pant 100P may include a backsheet 136. The backsheet 136 may also define the outer, garment facing surface 134 of the chassis 102. The backsheet 136 may also comprise a woven or nonwoven material, polymeric films such as thermoplastic films of polyethylene or polypropylene, and/or a multi-layer or composite materials comprising a film and a nonwoven material. The backsheet may also comprise an elastomeric film. An example backsheet 136 may be a polyethylene film having a thickness of from about 0.012 mm (0.5 mils) to about 0.051 mm (2.0 mils). Further, the backsheet 136 may permit vapors to escape from the absorbent core (i.e., the backsheet is breathable) while still preventing exudates from passing through the backsheet 136.

Also described above, the diaper pant 100P may include a topsheet 138. The topsheet 138 may also define all or part of the inner, wearer facing surface 132 of the chassis 102. The topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art. Topsheets 138 may be selected from high loft nonwoven topsheets, apertured film topsheets and apertured nonwoven topsheets. Exemplary apertured films may include those described in U.S. Pat. Nos. 5,628,097; 5,916,661; 6,545,197; and 6,107,539, all of which are incorporated by reference herein.

As mentioned above, the diaper pant 100P may also include an absorbent assembly 140 that is joined to the chassis 102. As shown in FIG. 2A, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

As previously mentioned, the diaper 100P may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1, all of which are incorporated by reference herein.

As mentioned above, diaper pants may be manufactured with a ring-like clastic belt 104 and provided to consumers in a configuration wherein the front waist region 116 and the back waist region 118 are connected to each other as packaged, prior to being applied to the wearer. As such, diaper pants may have a continuous perimeter waist opening 110 and continuous perimeter leg openings 112 such as shown in FIG. 1. The ring-like elastic belt may be formed by joining a first elastic belt to a second elastic belt with a permanent side scam or with an openable and reclosable fastening system disposed at or adjacent the laterally opposing sides of the belts.

As previously mentioned, the ring-like elastic belt 104 may be defined by a first elastic belt 106 connected with a second elastic belt 108. As shown in FIGS. 2A and 2B, the first elastic belt 106 extends between a first longitudinal side edge 111a and a second longitudinal side edge 111b and defines first and second opposing end regions 106a, 106b and a central region 106c. And the second clastic 108 belt extends between a first longitudinal side edge 113a and a second longitudinal side edge 113b and defines first and second opposing end regions 108a, 108b and a central region 108c. As measured in an extended state, the distance between the first longitudinal side edge 111a and the second longitudinal side edge 111b defines the pitch length, PL, of the first clastic belt 106, and the distance between the first longitudinal side edge 113a and the second longitudinal side edge 113b defines the pitch length, PL, of the second elastic belt 108. The central region 106c of the first clastic belt is connected with the first waist region 116 or first end region 116a of the chassis 102, and the central region 108c of the second clastic belt 108 is connected with the second waist region 118 or second end region 118a of the chassis 102. As shown in FIG. 1, the first end region 106a of the first clastic belt 106 is connected with the first end region 108a of the second clastic belt 108 at first side seam 178, and the second end region 106b of the first clastic belt 106 is connected with the second end region 108b of the second clastic belt 108 at second side seam 180 to define the ring-like clastic belt 104 as well as the waist opening 110 and leg openings 112. It is to be appreciated that the first belt 106 and the second belt 108 may be permanently or refastenably connected with each other at the first side seam 178 and the second side scam 180. The side seams 178, 180 may comprise a permanent bond, such as a thermal, pressure, or adhesive bond, or may be a releasable bond, such as a mechanical or cohesive fastener.

As shown in FIGS. 2A and 2B, the first clastic belt 106 also defines an outer laterally extending edge 107a and an inner laterally extending edge 107b, and the second elastic belt 108 defines an outer laterally extending edge 109a and an inner laterally extending edge 109b. The outer edge 107a of the first belt 106 is positioned longitudinally outward of the inner edge 107b, and the outer edge 109a of the second belt 108 is positioned longitudinally outward of the inner edge 109b. As such, as shown in FIG. 1, a perimeter edge 112a of one leg opening may be defined by portions of the inner laterally extending edge 107b of the first elastic belt 106, the inner laterally extending edge 109b of the second clastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100P.

It is to be appreciated that the first elastic belt 106 and the second elastic belt 108 may define different sizes and shapes. In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168 that extend along non-linear or curved paths that may correspond with the curved contours of the inner lateral edges 107b, 109b.

FIG. 2C shows a configuration wherein the first elastic belt 106 and the second elastic belt 108 both define generally rectangular shapes. For example, as shown in FIG. 2C, the outer laterally extending edge 107a of the first elastic belt 106 may comprise a lateral width of W1D and the inner laterally extending edge 107b may comprise a lateral width of W1P, wherein W1D and W1P are equal or substantially equal. In addition, the outer laterally extending edge 109a of the second elastic belt 108 may comprise a lateral width of W2D and the inner laterally extending edge 109b may comprise a lateral width of W2P, wherein W2D and W2P are equal or substantially equal.

In some configurations, at least one of the first elastic belt 106 and the second elastic belt 108 may comprise lateral edges having different lengths. For example, FIG. 2D shows a configuration wherein the first elastic belt 106 defines a generally rectangular shape, such as described with reference to FIG. 2C, and wherein the outer laterally extending edge 109a of the second clastic belt 108 and the inner laterally extending edge 109b have different lengths. As shown in FIG. 2D, the outer laterally extending edge 109a of the second elastic belt 108 may comprise a lateral width of W2D and the inner laterally extending edge 109b may comprise a lateral width of W2P, wherein W2D is greater than W2P.

In some configurations, both the first elastic belt 106 and the second elastic belt 108 may comprise lateral edges having different lengths. For example, FIG. 2E shows a configuration wherein the outer laterally extending edge 107a of the first elastic belt 106 and the inner laterally extending edge 107b have different lengths, and wherein the outer laterally extending edge 109a of the second elastic belt 108 and the inner laterally extending edge 109b have different lengths. As shown in FIG. 2E, the outer laterally extending edge 107a of the first elastic belt 107 may comprise a lateral width of W1D and the inner laterally extending edge 107b may comprise a lateral width of W1P, wherein W1D is greater than W1P, and wherein the outer laterally extending edge 109a of the second elastic belt 108 may comprise a lateral width of W2D and the inner laterally extending edge 109b may comprise a lateral width of W2P, wherein W2D is greater than W2P.

With reference to FIGS. 2C-2E, the first elastic belt 106 may define a longitudinal length LT1 extending between outer laterally extending edge 107a and the inner laterally extending edge 107b, and the second clastic belt 108 may define a longitudinal length LT2 extending between outer laterally extending edge 109a and the inner laterally extending edge 109b. In some configurations, LT1 may be equal to LT2. In some configurations, LT1 may be less or greater than LT2. With continued reference to FIGS. 2C-2E, in some configurations, W1D may be equal to W1P, or W1D may be different than W1P. In some configurations, W2D may be equal to W2P, or W2D may be different than W2P. In some configurations, W1D and/or W1P may be equal to or different W2D and/or W2P.

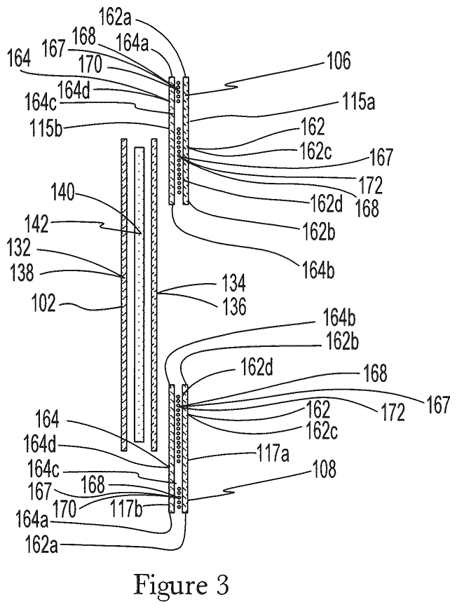

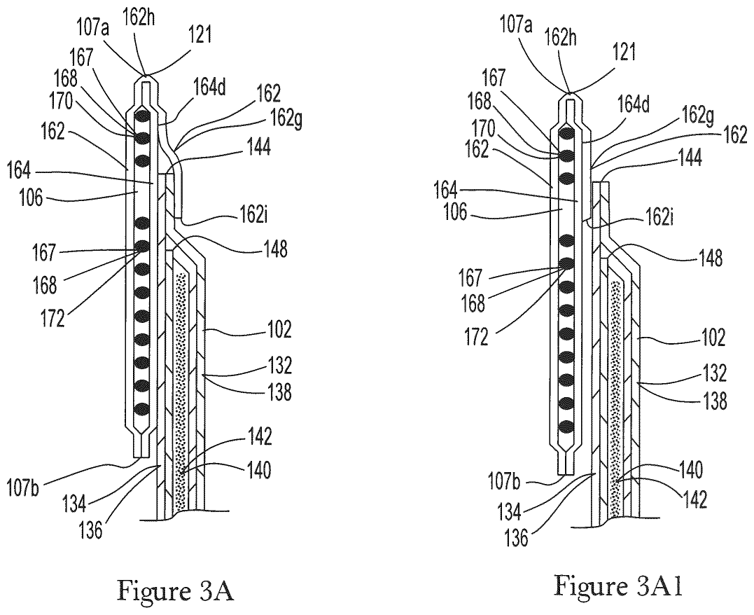

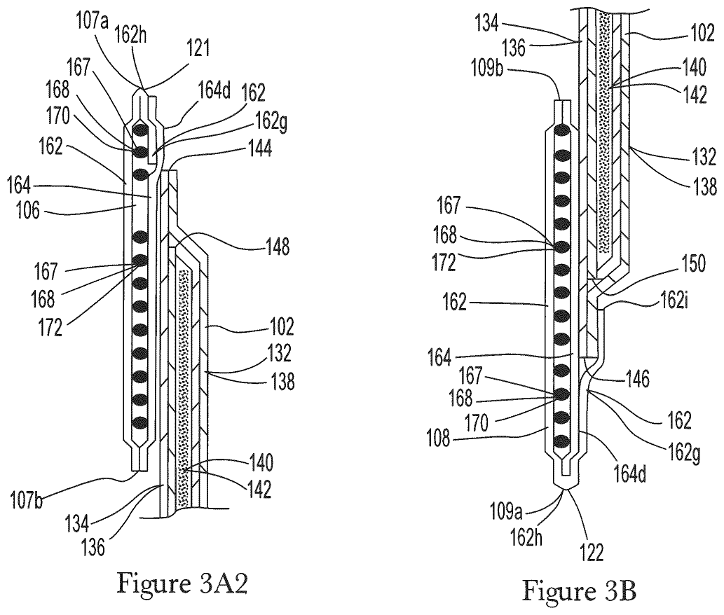

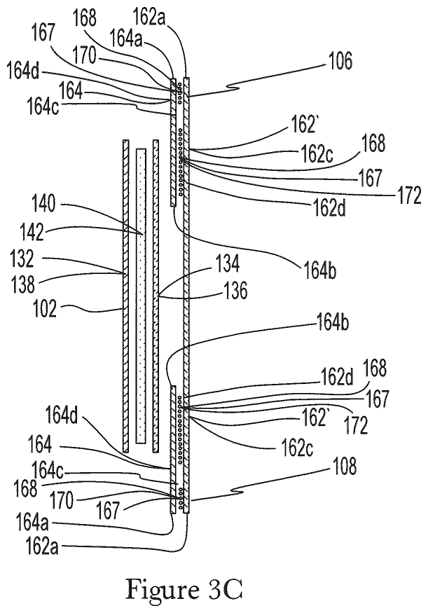

With reference to FIGS. 2A, 2B, and 3, the first elastic belt 106 and the second elastic belt 108 may also each include a first substrate 162 and a second substrate 164. The first substrates 162 may be oriented to define at least a portion of a garment facing surface 115a of the first elastic belt 106 and a garment facing surface 117a the second clastic belt 108, and the second substrates 164 may be oriented to define at least a portion of a wearer facing surface 115b of the first elastic belt 106 and a wearer facing surface 117b of the second elastic belt 108. The first substrate 162 may extend from a proximal edge 162b to a distal edge 162a for a maximum length L1, and the second substrate 164 may extend from a proximal edge 164b to a distal edge 164a for a maximum length L2. It is to be appreciated that the distal edge 162a and/or the proximal edge 162b of the first substrate 162 may be straight and/or curved and/or may be parallel or unparallel to each other. It is also to be appreciated that the distal edge 164a and/or the proximal edge 164b of the second substrate 164 may be straight and/or curved and/or may be parallel or unparallel to each other. As such, the maximum length L1 refers to the longest distance extending longitudinally between the distal edge 162a and the proximal edge 162b of the first substrate 162, and the maximum length L2 refers to the longest distance extending longitudinally between the distal edge 164a and the proximal edge 164b of the second substrate 164. In some configurations, L1 may be equal to, less than, or greater than L2. In some configurations, L1 may be equal to or less than LT1, and L2 may be equal to or less than LT2. In some configurations, the distal edge 162a of the first substrate 162 may define at least a portion of the front waist edge 121 and/or at least a portion of back waist edge 122, and/or the distal edge 164a of the second substrate 164 may define at least a portion of the front waist edge 121 and/or at least a portion of back waist edge 122. As such, in some configurations, the distal edge 162a of the first substrate 162 and/or the distal edge 164a of the second substrate 164 may define at least a portion of the waist opening 110.

It is also to be appreciated that the first substrate 162 and/or the second substrate 164 may extend continuously from the first belt 106 to the second belt 108. For example, the first substrate 162 may be configured to define a continuous outer cover 162′ that extends contiguously from the first waist edge 121 to the second waist edge 122, such as shown in FIGS. 1A, 2F, and 3C. It is also to be appreciated that diaper pants 100P with continuous outer covers, such as shown in FIGS. 1A, 2F, and 3C may also be configured to include various aspects of the frangible pathways and fastener components discussed herein.

It is to be appreciated that the first substrate 162 and the second substrate 164 may define various lateral widths that may or may not be equal. For example, as shown in FIG. 2B, the first substrate 162 may extend laterally between a first longitudinal edge 162e and a second longitudinal edge 162f to define a first lateral width W1, and the second substrate 164 may extend laterally between a first longitudinal edge 164e and a second longitudinal edge 164f to define a second lateral width W2.

In some configurations, the proximal edge 162b of the first substrate 162 and/or the proximal edge 164b of the second substrate 164 may extend laterally across the backsheet 136. As shown in FIGS. 2A-3, the first substrate 162 includes a garment facing surface 162c and an opposing wearer facing surface 162d, and the second substrate 164 includes a garment facing surface 164c and an opposing wearer facing surface 164d.

In some configurations, the first elastic belt 106 and/or the second elastic belt 108 may include a folded portion of at least the first substrate 162 and/or the second substrate 164. For example, as shown in FIGS. 3A and 3B, the first elastic belt 106 and/or the second elastic belt 108 may include a folded portion 162g of the first substrate 162 extending longitudinally between a fold line 162h in the first substrate 162 and a lateral edge 162i. As such, the folded portion 162g of the first substrate 162 may be connected with the wearer facing surface 164d of the second substrate 164. In some configurations, the folded portion 162g of the first substrate 162 may also be connected with and/or overlap the chassis 102. In some configurations, the folded portion 162g of the first substrate 162 may also be connected with the wearer facing surface 162d of the first substrate 162. In some configurations, a portion of the folded portion 162g of the first substrate 162 may be left unbonded to the chassis 102 and/or the second substrate 164, forming a pocket having an opening oriented toward the lateral centerline 162c of the chassis 102. In another example, the first elastic belt 106 and/or the second elastic belt 108 may include a folded portion of the second substrate 164 extending longitudinally between a fold line in the second substrate 164 and a lateral edge. As such, the folded portion of the second substrate 164 may be connected with the garment facing surface 162c of the first substrate 162. As such, in some configurations, a fold line of the first substrate 162 and/or a fold line of the second substrate 164 may define at least a portion of the waist opening 110. It is to be appreciated that various waist configurations may be utilized. For example, as shown in FIG. 3A1, the folded portion 162g may be sandwiched between the second substrate 164 and the backsheet 136. In another example shown in FIG. 3A2, the second substrate 164 may be sandwiched between the folded portion 162g and the backsheet 136. Although FIGS. 3A1 and 3A2 show configurations of the first belt 106, it is to be appreciated that such configurations may be applied with the second belt 108.

It is to be appreciated that the first elastic belt 106 and the second elastic belt 108 may comprise the same materials and/or may have the same structure. In some embodiments, the first clastic belt 106 and the second elastic belt may comprise different materials and/or may have different structures. It should also be appreciated that components of the first elastic belt 106 and the second elastic belt 108, such as the first substrate 162, and/or second substrate 164 may be constructed from various materials. For example, the first and/or second belts may include a first substrate 162, and/or second substrate 164 that may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some configurations, the first and/or second belts may include a first substrate 162, and/or second substrate 164 comprising a nonwoven web of synthetic fibers, and may include a stretchable nonwoven. In some configurations, the first and second clastic belts may include an inner hydrophobic, non-stretchable nonwoven material and an outer hydrophobic, non-stretchable nonwoven material. It is to be appreciated that the belts may configured in various ways, such as disclosed for example, in U.S. Patent Publication No. 2022/0142828 A1, which are both incorporated by reference.

Elastic material 167 may be positioned between the wearer facing surface 162d of the first substrate 162 and the garment facing surface 164c of the second substrate 164. It is to be appreciated that the elastic material 167 may include one or more elastic elements such as strands, ribbons, clastic films, or panels extending along the lengths of the elastic belts. As shown in FIGS. 2A and 3, the elastic material 167 may include a plurality of elastic strands 168. In some configurations, the elastic material 167 may be an elastic film used to form a zero-strain elastic laminate comprising an elastic film bonded to one or more nonwoven layers and subsequently subjected to mechanical deformation or activation sufficient to weaken the nonwoven layer(s) and enable the laminate to stretch and recover elastically.

It is also to be appreciated that the first substrate 162, second substrate 164, and/or elastic material 167 of the first elastic belt 106 and/or second elastic belt 108 may be bonded together and/or with other components, such as the chassis 102, with adhesive and/or mechanical bonds. It is to be appreciated that adhesive and mechanical bonding methods may be utilized alone or in combination with each other.

In some configurations, adhesive may be applied to at least one of the first substrate 162, second substrate 164, and/or elastic material 167 when being combined to form the first elastic belt 106 and/or second elastic belt 108. In some configurations, mechanical bonding devices may apply mechanical bonds to the to at least one of the first substrate 162, second substrate 164, and/or clastic material 167 when being combined to form the first clastic belt 106 and/or second elastic belt 108. Such mechanical bonds may be applied with heat, pressure, and/or ultrasonic devices. In some configurations, mechanical bonding devices may apply bonds that bond the first substrate 162, second substrate 164, and/or elastic material 167 together and/or may act to trap or immobilize discrete lengths of the contracted elastic strands in the first elastic belt 106 and/or second elastic belt 108.

It is to be appreciated that components of the first elastic belt 106 and/or the second elastic belt 108 may be assembled in various ways and various combinations to create various desirable features that may differ along the lateral width and/or longitudinal length of the first elastic belt 106 and/or the second elastic belt 108. Such features may include, for example, Dtex values, bond patterns, aperture arrangements, elastic positioning, Average Dtex values, Average Pre-Strain values, rugosity frequencies, rugosity wavelengths, height values, and/or contact area. It is to be appreciated that differing features may be imparted to various components, such as for example, the first substrate 162, second substrate 164, and elastic material 167 before and/or during stages of assembly of the first elastic belt 106 and/or the second elastic belt 108.

It is to be appreciated that the first elastic belt 106 and/or the second elastic belt 108 may include various configurations of belt elastic materials 167 arranged in relation to each other and to the first substrate 162, and the second substrate 164. As discussed above, the clastic material 167 may include configurations of one or more elastic elements such as strands, ribbons, films, or panels positioned in various arrangements. In some configurations, the elastic material 167 may comprise various elastics, elastic features and arrangements, and processes for assembly, and that the first substrate 162, second substrate 164, and/or elastic material 167 may be bonded together with various methods and apparatuses to create various elastomeric laminates, such as described in 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0298586 A1; 2019/0070042 A1; 2018/0168878 A1; 2018/0168877 A1; 2018/0168880 A1; 2018/0170027 A1; 2018/0169964 A1; 2018/0168879 A1; 2018/0170026 A1; 2019/0070041 A1; 2021/0282797 A1; and 2021/0275362 A1, which are all incorporated by reference. It is also to be appreciated the elastic materials 167 herein may be configured with identical or different colors in various different locations on the first clastic belt 106 and/or the second elastic belt 108.

In some configurations, the clastic material 167 may be configured as clastic strands 168 disposed at a constant interval in the longitudinal direction. In other embodiments, the clastic strands 168 may be disposed at different intervals in the longitudinal direction. In some configurations, the Dtex values of the clastic strands 168 may be constant or varied along the longitudinal direction. In some configurations, the clastic material 167 in a stretched condition may be interposed and joined between uncontracted substrate layers. When the elastic material 167 is relaxed, the clastic material 167 returns to an unstretched condition and contracts the substrate layers. The elastic material 167 may provide a desired variation of contraction force in the area of the ring-like elastic belt. It is to be appreciated that the chassis 102 and elastic belts 106, 108 may be configured in different ways other than as depicted in attached Figures. It is also to be appreciated that the elastic material 167 material may be joined to the substrates continuously or intermittently along the interface between the clastic material 167 material and the substrates. In some configurations, the clastic strands 168 may be in the form of extruded elastic strands, which may also be bonded with the first substrate 162 and/or second substrate 164 in a pre-corrugated configuration, such as disclosed for example in U.S. Pat. No. 5,681,302, which is incorporated by reference herein.

As discussed above for example with reference to FIGS. 2A and 3, the clastic material 167 discussed herein may be in the form of clastic strands 168. In some configurations, the clastic strands 168 may be parallel with each other and/or with the lateral axis 126. It is to be appreciated that the first clastic belt 106 and/or second clastic belt 108 may be configured to include various quantities of clastic strands 168. In some configurations, clastic strands 168 may be grouped in pairs. In some configurations, the first elastic belt 106 and/or second elastic belt 108 may comprise from about 10 to about 1500 clastic strands 168. It is also to be appreciated that clastic strands 168 herein may comprise various Dtex values, strand spacing values, and pre-strain values and such clastic strands 168 may utilized with other elastic strands to create first and second clastic belts 106, 108 comprising elastic strands 168 in various combinations of Dtex values, strand spacing values, and pre-strain values. For example, in some configurations, the Average-Dtex of one or more clastic strands 168 may be greater than 500. In some configurations, the Average-Dtex of one or more elastic strands 168 may be from about 10 to about 1500, specifically reciting all 1 Dtex increments within the above-recited range and all ranges formed therein or thereby. In some configurations, a plurality of clastic strands 168 may comprise an Average-Strand-Spacing of less than or equal to 4 mm. In some configurations, a plurality of clastic strands 168 may comprise an Average-Strand-Spacing from about 0.25 mm to about 4 mm, specifically reciting all 0.01 mm increments within the above-recited range and all ranges formed therein or thereby. In some configurations, a plurality of elastic strands 168 may comprise an Average-Strand-Spacing of greater than 4 mm. In some configurations, the Average-Pre-Strain of each of a plurality of elastic strands may be from about 50% to about 400%, specifically reciting all 1% increments within the above-recited range and all ranges formed therein or thereby. In some configurations, the elastic strands 168 comprise an Average-Strand-Spacing from about 0.25 mm to about 4 mm and an Average-Dtex from about 10 to about 500. In some configurations, the elastic strands 168 may comprise an Average-Pre-Strain from about 75% to about 300%.

In some configurations, a first plurality of elastic strands may comprise a first Average-Pre-Strain from about 75% to about 300%, and a second plurality of elastic strands may comprise a second Average-Pre-Strain that is greater than first Average-Pre-Strain. In some configurations, a first plurality of clastic strands comprises an Average-Strand-Spacing from about 0.25 mm to about 4 mm and an Average-Dtex from about 10 to about 500; and a second plurality of elastic strands may comprise an Average-Strand-Spacing greater than about 4 mm and an Average-Dtex greater than about 450.

In some configurations, such as shown in FIG. 2A, the elastic strands 168 may be referred to herein as outer waist elastics 170 and inner waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. Some elastic strands 168, such as the inner waist elastics 172, may be configured with discontinuities in areas, such as for example, where the first and second elastic belts 106, 108 overlap portions of the chassis 102, such as the absorbent assembly 140.

As shown in FIG. 2A, the first elastic belt 106 and/or the second elastic belt 108 may be configured with low-stretch zones 701 and high-stretch zones 703. The first elastic belt 106 and/or the second clastic belt 108 may include a first high-stretch zone 703a and a second high-stretch zone 703b separated laterally by a low-stretch zone 701. Portions of the chassis 102, such as the backsheet 136 and absorbent assembly 140, may be connected with the first clastic belt 106 and/or the second clastic belt 108 in the low-stretch zones 701 in the first waist region 116 and/or the second waist region 118. The high-stretch zones 703 are elasticated by the elastic material 167, such as the clastic strands 168, 172; and the low-stretch zones 701 may comprise cut lines separating the clastic material 167, such as the clastic strands 168, 172. In some configurations, the clastic material 167 may be cut in an unbonded region where the clastic material is not bonded with first substrate 162 and the second substrate 164. Thus, the clastic material 167 retracts from the unbonded region and form low-stretch zone 701. In some configurations, the clastic material 167 may be cut into several discrete pieces. In turn, the low-stretch zones 701 define regions of the first clastic belt 106 and/or the second elastic belt 108 that have relatively less elasticity than the high-stretch zones 703. The discrete clastic material 167 that has been cut and which are elastically contracted do not add any substantial amount of elastication to the low-stretch zone 701. As such, upon application of a force, the high-stretch zones 703 will elongate more than the low-stretch zones 701. As provided above, the terms “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. In some configurations, the first clastic belt 106 and/or the second clastic belt 108 may be configured with high-stretch zones 703 that are clastic and may be configured with low-stretch zones 701 that are not elastic or “inelastic.”

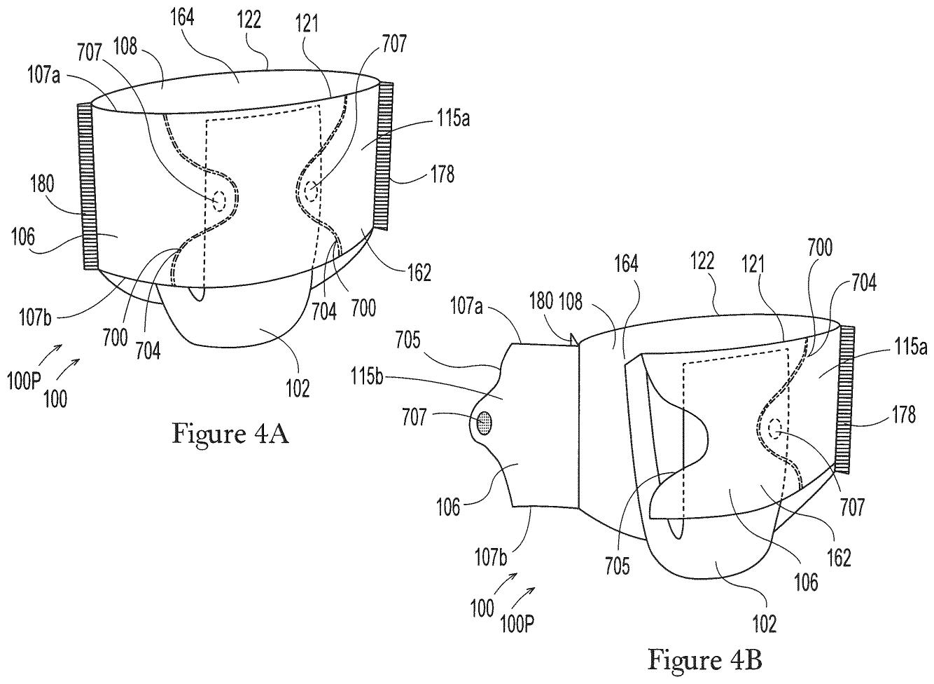

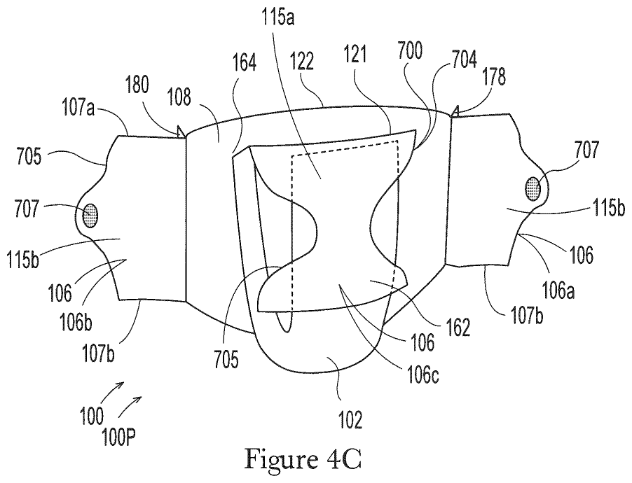

As discussed above, the diaper pants 100P described with reference to FIGS. 1-3C may include one or more frangible pathways in the first belt 106 and/or the second belt 108. For example, FIGS. 4A-4C show an example diaper pant 100P with a first belt 106 that includes frangible pathways 700. The frangible pathways 700 may be configured to allow the first clastic belt 106 to be relatively easily torn along the frangible pathway 700, such as when removing the diaper pant 100P from a wearer. FIG. 4B shows a view of the diaper pant 100P from FIG. 4A, illustrating the first belt 106 after having been torn along the frangible pathway 700 through both the outer longitudinal outer laterally extending edge 107a and the inner laterally extending edge 107b of the first belt 106. As such, the first elastic belt 106 shown in FIG. 4B is separated by opposing tear lines 705. It is to be appreciated the first clastic belt 106 may be torn along both frangible pathways 700 in FIGS. 4B. For example, FIG. 4C shows the diaper pant of FIG. 4A showing the front belt having been torn along two frangible pathways 700. As shown in FIG. 4C, the central region 106c of the first clastic belt 106 may remain bonded with the chassis 102 after separating the first and second opposing end regions 106a, 106b from the central region 106c by tearing the clastic belt 106 along the frangible pathways 700.

As discussed in more detail below, the frangible pathways 700 comprise a plurality of lines of weakness 704 configured such that all clastic strands 168 in the first elastic belt 106 are severed at least once in the frangible pathway 700. Severing the elastic strands 168 in the frangible pathway 700 helps make it relatively easier to tear the first elastic belt 106 along the frangible pathway 700. For example, when the clastic strands 168 are severed, the first substrate 162 and second substrate 164 of the first clastic belt 106 need only need to be torn without having to also tear uncut clastic strands 168. It is to be appreciated that the diaper pant 100P may include various quantities of frangible pathways 700 that may be: positioned in various locations; define various shapes; and extend for various lengths. For example, the first elastic belt 106 may comprise a first belt length defined by a longitudinal distance between the proximal edge 107b and the distal edge 107a, and the frangible pathway 700 may extend for a total length from an outermost edge of a line of weakness 704 nearest the proximal edge 107b of the first belt 106 to an outermost edge of a line of weakness 704 nearest the distal edge 107a of the first belt 106. In some configurations, the frangible pathway 700 may extend for a total length that is greater than, equal to, or less than the first belt length. In some configurations, the lines of weakness 704 may extend for a length from a first end to a second end, and a sum of the all the lengths of lines of weakness 704 in the frangible pathway 700 may be greater than the frangible pathway total length.

In some configurations, diaper pants 100P may be configured such that one or both of the first elastic belt 106 and the second clastic belt 108 include one or more frangible pathways 700. The frangible pathways 700 may be positioned in various locations on the first and second clastic belts 106, 108. For example, such as shown in FIGS. 4A-4C, frangible pathways 700 may extend to overlap with the chassis 102. In some configurations, the frangible pathways 700 may extend in straight lines and/or may be curved and/or have curved portions. In some configurations, the frangible pathways 700 may extend longitudinally for the entire length or less than the entire length of the first belt 106 and/or second belt 108. In some configurations, frangible pathways 700 may be positioned partially or entirely laterally between the first and second side scams 178, 180 and the chassis 102.

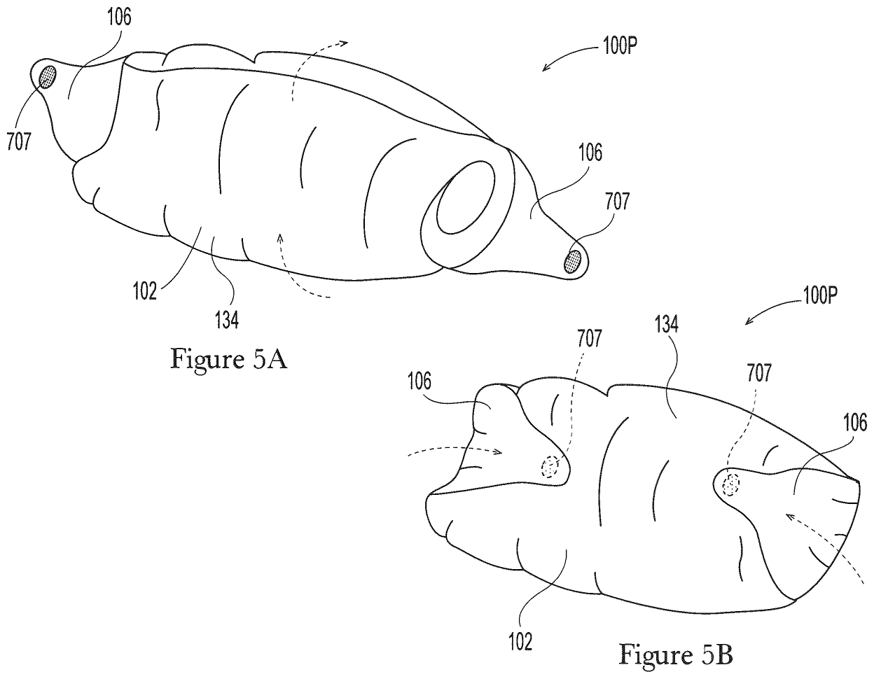

In some configurations, the frangible pathways 700 may be configured and/or positioned to provide access to and/or function with other features, such as disposal features. For example, the diaper pant 100P shown in FIGS. 4A-4C includes fastener components 707 positioned on the wearing facing surface 115b of the first clastic belt 106. In some configurations, the fastener components 707 may be positioned between the first clastic belt 106 and the chassis 102. The fastener component 707 may be configured to refastenably connect with other portions of the diaper pant 100P, such as for example, the garment facing surfaces of the first elastic belt 106, the second clastic belt 108, or the chassis 102. As such, once the first clastic belt 106 is torn along the frangible pathways 700, the diaper pant 100P may be removed from a wearer and rolled or folded up for disposal, and the fastener component 707 may be connected with another portion of the diaper pant 100P to help maintain the diaper pant 100P in a disposal configuration. For example, FIG. 4C shows a diaper pant 100P after tearing the first elastic belt 106 along two frangible pathways. FIG. 5A shows the diaper pant 100P of FIG. 4C with the chassis 102 being rolled up onto itself in a longitudinal direction. And FIG. 5B shows the diaper pant 100P of FIG. 5A with fastener components 707 refastenably connected with the backsheet 136 of the chassis 102 to maintain the diaper pant 100P in a disposal configuration. In some configurations, when tearing the clastic belt along the frangible pathway 700, the tearing process may begin by tearing from the outer edge 107a or the inner edge 107b of the clastic belt 106. As discussed in more detail below, in some configurations, the first elastic belt 106 may also include an opening, such as a slit located adjacent to or in the proximity of the fastener component 707 and the weakened region 700 to help facilitate starting to tear the frangible pathway 700 in a region of the elastic belt 106 longitudinally between the outer edge 107a and the inner edge 107b.

It is also to be appreciated that the frangible pathways 700 may comprise lines of weakness 704 that are: configured in various ways; positioned in various locations and orientations relative to each other; defined by various shapes; and extend for various lengths. For example, in some configurations, the lines of weakness 704 comprise discrete cut lines that penetrate through at least one or all the layers of the elastic belt 106. In some configurations, the lines of weakness 704 comprise discrete bonds wherein materials of the first substrate and the second substrate are fused together. In some configurations, the lines of weakness 704 may be linear, curvilinear, or have a regular or irregular geometry and may comprise one or more of a perforation, a bond, an aperture, or a mechanically thinned region of a material such as a nonwoven, or a combination thereof. It is also to be appreciated that the lines of weakness 704 can be formed with different lengths and spacings to achieve different separation forces.

As discussed above, absorbent articles 100, such as diaper pants 100P, may be configured with frangible pathways 700 comprising lines of weakness 704 arranged in various ways to help improve a caregiver's ability to remove a soiled diaper pant 100P from a wearer without having to remove a soiled diaper pant from a wearer by sliding the soiled diaper pant down the wearer's legs. As discussed above, the frangible pathways 700 may be configured to allow the first elastic belt 106 and/or the second clastic belt 108 to be relatively easily torn along the frangible pathway 700, such as when removing the diaper pant 100P from a wearer. In addition, the frangible pathways 700 may also be configured to provide access to fastener components 707 that may be used to help hold a soiled product in a disposal configuration. The following provides a discussion of example implementations of frangible pathways 700 on diaper pants 100P in the context of the above description of various details of absorbent articles 100, fastener components 707, frangible pathways 700, and lines weakness 704. It is to be appreciated that discussions of frangible pathways 700 in the first elastic belt 106 herein may also apply to frangible pathways 700 in the second clastic belt 108.

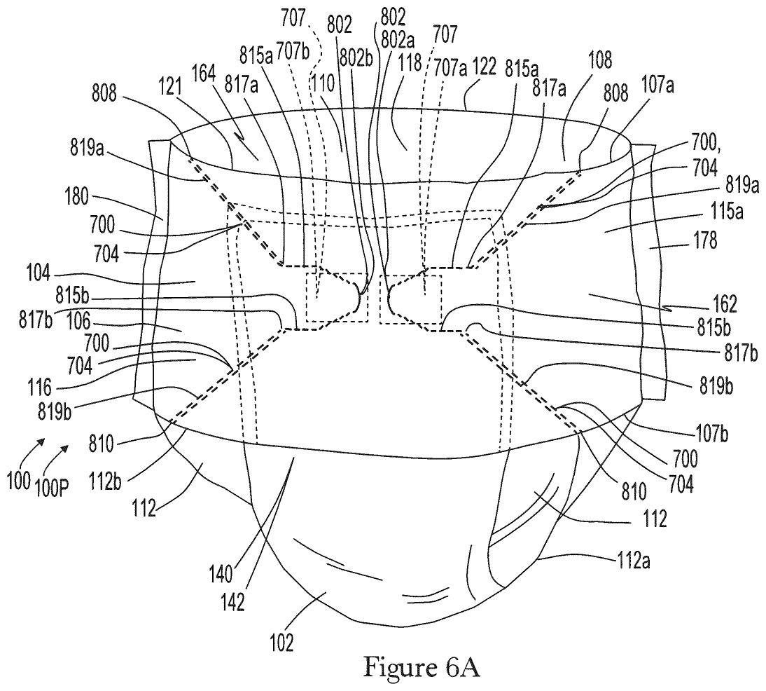

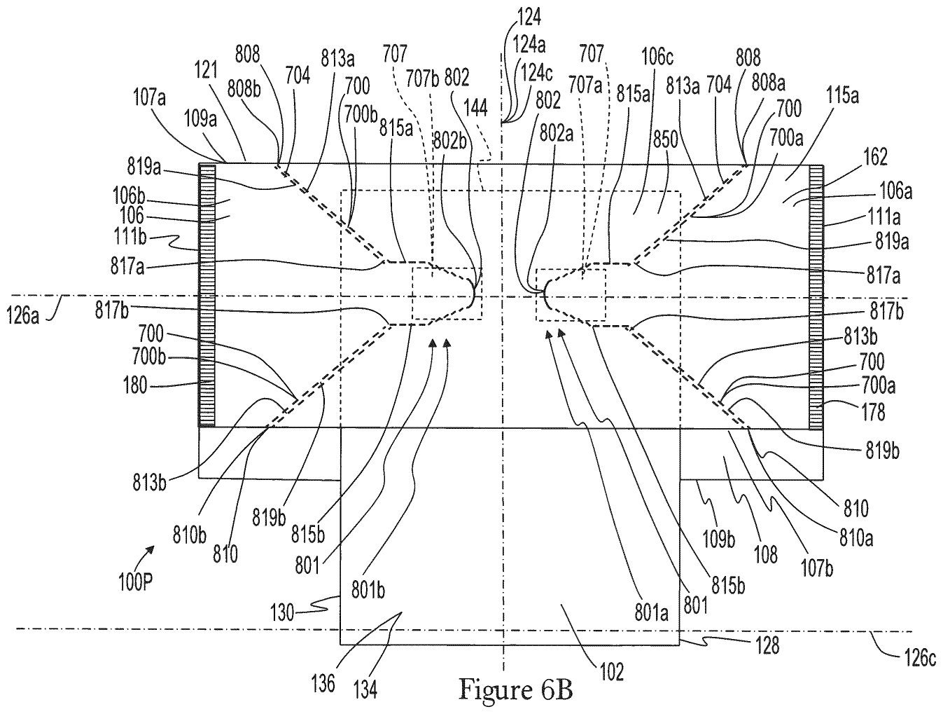

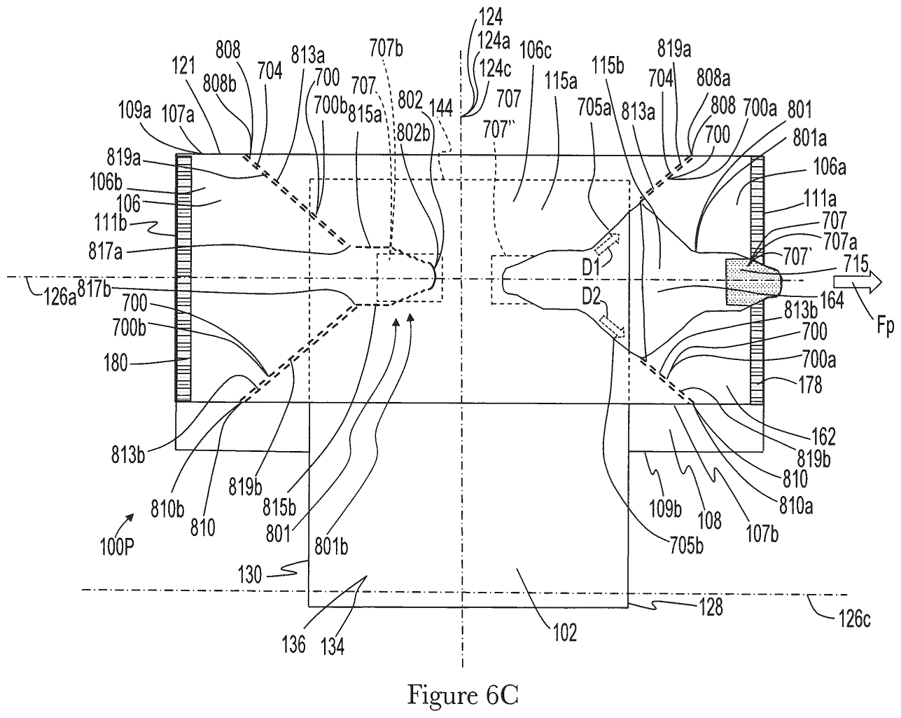

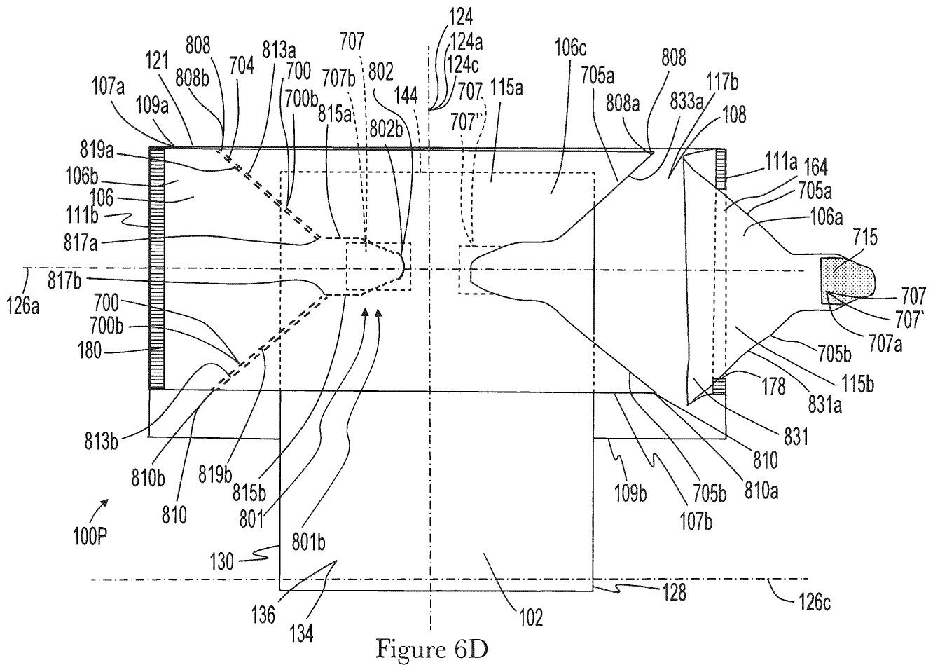

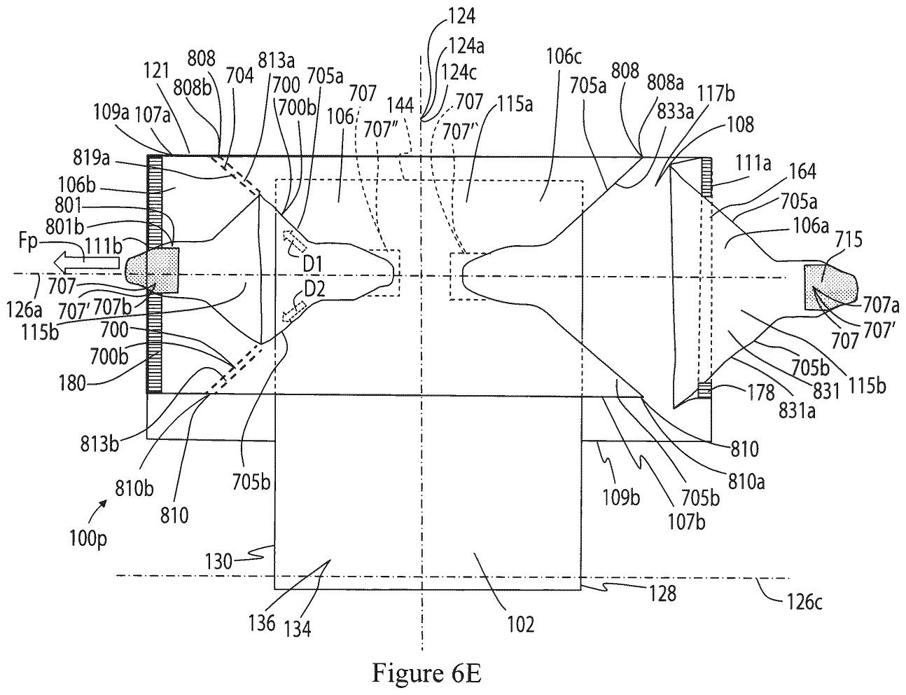

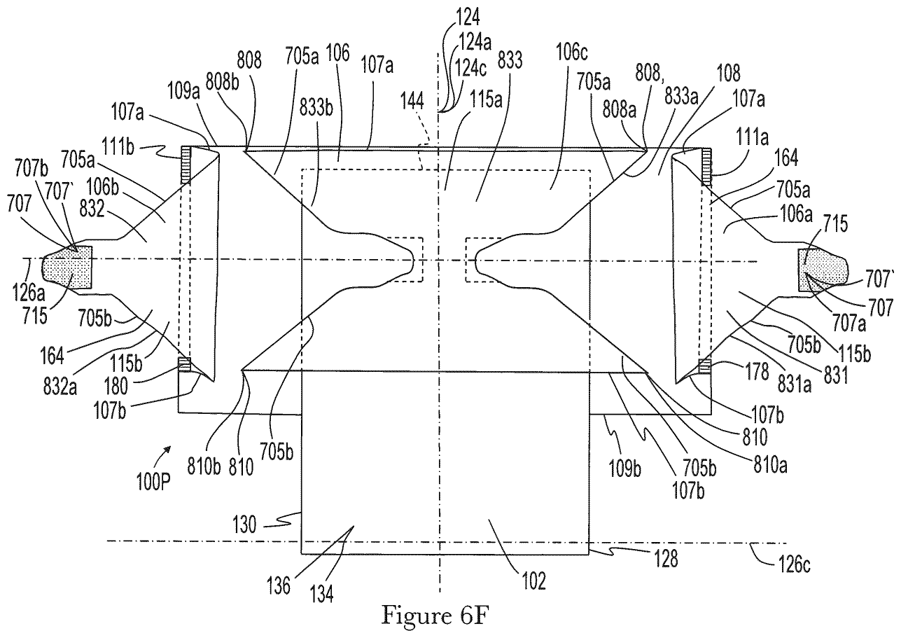

It is to be appreciated that frangible pathways 700 may be positioned in various locations and/or orientations relative to other components of the absorbent article 100 and/or may be configured to function in various ways to help facilitate removal of diaper pant from a wearer. For example, the diaper pant 100P shown in FIGS. 6A and 6B may include one or more frangible pathways 700 extending between a distal terminus 808 on the outer edge 107a of the first belt 106 and a distal terminus 810 on the inner edge 107b of the first belt 106. As illustrated in FIGS. 6A and 6B, the diaper pant 100P includes a first frangible pathway 700a and a second frangible pathway 700b in the first belt 106. The first frangible pathway 700a may extend between a first distal terminus 808a on the outer edge 107a of the first belt 106 and a first proximal terminus 810a on the inner edge 107b of the first belt 106. And the second frangible pathway 700b may extend between a second distal terminus 808b on the outer edge 107a of the first belt 106 and a second proximal terminus 810b on the inner edge 107b of the first belt 106. It is to be appreciated that the first and second frangible pathways 700a, 700b may comprise lines of weakness 704 as described above.

It is to be appreciated that the first distal terminus 808a and the second distal terminus 808b may be located in various lateral positions on the outer edge 107a of the first belt 106. For example, in some configurations, the first distal terminus 808a and/or the second distal terminus 808b may be positioned in the central region 106c of the first belt 106. In some configurations, the first distal terminus 808a and/or the second distal terminus 808b may be positioned laterally between the first longitudinal edge 128 and the second longitudinal edge 130 of the chassis 102. In some configurations, the first distal terminus 808a and/or the second distal terminus 808b may be positioned in the first end region 106a and/or the second end region 106b of the first belt 106. In some configurations, the first distal terminus 808a and/or the second distal terminus 808b may be positioned laterally outboard of the first longitudinal edge 128 and the second longitudinal edge 130 of the chassis 102. In some configurations, the first distal terminus 808a and/or the second distal terminus 808b may be positioned laterally between the first longitudinal edge 128 of the chassis 102 and the first side seam 178 and/or may be positioned laterally between the second longitudinal edge 130 of the chassis 102 and the second side seam 180. In some configurations, the first distal terminus 808a may be laterally aligned with the first longitudinal edge 128 of the chassis 102 or the first longitudinal side edge 111a of the first belt 106. In some configurations, the first distal terminus 808a may be positioned laterally between the first longitudinal edge 128 of the chassis 102 and the first longitudinal side edge 111a of the first belt 106. In some configurations, the second distal terminus 808b may be laterally aligned with the second longitudinal edge 130 of the chassis 102 or the second longitudinal side edge 111b of the first belt 106. In some configurations, the second distal terminus 808b may be positioned laterally between the second longitudinal edge 130 of the chassis 102 and the second longitudinal side edge 111b of the first belt 106.

It is also to be appreciated that the first proximal terminus 810a and the second proximal terminus 810b may be located in various lateral positions on the inner edge 107b of the first belt 106. For example, in some configurations, the first proximal terminus 810a and/or the second proximal terminus 810b may be positioned in the central region 106c of the first belt 106. In some configurations, the first proximal terminus 810a and/or the second distal terminus 810b may be positioned laterally between the first longitudinal edge 128 and the second longitudinal edge 130 of the chassis 102. In some configurations, the first proximal terminus 810a and/or the second proximal terminus 810b may be positioned in the first end region 106a and/or the second end region 106b of the first belt 106. In some configurations, the first proximal terminus 810a and/or the second proximal terminus 810b may be positioned laterally outboard of the first longitudinal edge 128 and the second longitudinal edge 130 of the chassis 102. In some configurations, the first proximal terminus 810a and/or the second proximal terminus 810b may be positioned laterally between the first longitudinal edge 128 of the chassis 102 and the first side seam 178 and/or may be positioned laterally between the second longitudinal edge 130 of the chassis 102 and the second side seam 180. In some configurations, the first proximal terminus 810a may be laterally aligned with the first longitudinal edge 128 of the chassis 102 or the first longitudinal side edge 111a of the first belt 106. In some configurations, the first proximal terminus 810a may be positioned laterally between the first longitudinal edge 128 of the chassis 102 and the first longitudinal side edge 111a of the first belt 106. In some configurations, the second proximal terminus 810b may be laterally aligned with the second longitudinal edge 130 of the chassis 102 or the second longitudinal side edge 111b of the first belt 106. In some configurations, the second proximal terminus 810b may be positioned laterally between the second longitudinal edge 130 of the chassis 102 and the second longitudinal side edge 111b of the first belt 106.

As shown in FIG. 6B, the frangible pathways 700 may be configured to extend laterally inward from the from the distal terminus 808 and/or the proximal terminus 810. In turn, portions of the frangible pathway 700 may extend to define an angle that is less than 90 degrees with respect to the outer edge 107a and/or the inner edge 107b of the first belt 106. Thus, the frangible pathway may define an overall length that is greater than a longitudinal length LT1 of the first belt 106 and/or the longitudinal length LT2 of the second belt 108 discussed above with reference to FIGS. 2C-2E.

With continued reference to FIG. 6B, the first belt 106 may also comprise grip regions 801 providing a place where a user may grasp a portion of the first belt 106 and begin the process of tearing the first belt along the frangible pathway 700. The grip region 801 may comprise an accessibility opening 802 in the first belt 106 and may also comprise a fastener component 707 positioned adjacent the accessibility opening 802. The accessibility opening 802 may comprise slits and/or openings in the first belt 106 and may penetrate through some or all layers of the first belt 106. It is to be appreciated that such slits or openings may be curved and/or straight. The accessibility opening 802 may also be considered part of the frangible pathway 700.

As shown in FIG. 6B, the diaper pant 100P may include a first grip region 801a including a first accessibility opening 802a and second grip region 801b including a second accessibility opening 802b in the first belt 106. The first and second accessibility openings 802a, 802b may be positioned between the outer edge 107a and the inner edge 107b of the first belt 106. The first and second accessibility openings 802a, 802b may also be positioned in the central region 106c of the first belt 106 and may be positioned between the first longitudinal edge 128, the second longitudinal edge 130 of the chassis 102, and the first lateral edge 144 of the chassis 102. In addition, a first fastener component 707a may be positioned adjacent the first accessibility opening 802a, and a second fastener component 707a may be positioned adjacent the second accessibility opening 802a.

As discussed in more detail below, the accessibility opening 802 may help provide a caregiver or wearer access to and/or to grasp the fastener component 707 in the grip region 801 with a finger or thumb. The caregiver or user may then pull on grip region 801 to begin tearing the first belt 106 on the frangible pathway 700. In some configurations, tear lines may simultaneously propagate along the first tear zone 813a and the second tear zone 813b laterally outward from the central region 106c of the first belt 106 toward the distal terminus 808 and the proximal terminus 810. In some configurations, a tear line propagating along the first tear zone 813a and a tear line propagating along the second tear zone 813b may reach the distal terminus 808 and the proximal terminus 810, respectively, simultaneously or approximately simultaneously. It is also to be appreciated that some diaper pants 100P herein may be configured to include a frangible pathway 700 that extends through or around the fastener component 707 without an accessibility opening. In turn, a user may pinch and/or pull the belt where the frangible pathway 700 is located at or adjacent the fastener component 707 to initiate the tearing process along the frangible pathway 700.

With continued reference to FIG. 6B, the first frangible pathway 700a may comprise a first tear zone 813a extending from the first accessibility opening 802a to the first distal terminus 808a and a second tear zone 813b extending from the first accessibility opening 802a to the first proximal terminus 810a. The second frangible pathway 700b may comprise a first tear zone 813a extending from the second accessibility opening 802b to the second distal terminus 808b and a second tear zone 813b extending from the second accessibility opening 802b to the second proximal terminus 810b.

It is to be appreciated that the frangible pathways 700 may also comprise one or more functional zones. In turn, the frangible pathways 700 may comprise transition zones 817 that may operatively connect such zones to help facilitate propagation of a tear along the frangible pathway 700 from one zone to another zone. The lines of weakness in the transition zones may be of particular lengths and/or angles relative to lateral centerlines and row spacing to help provide desired propagation of material failure when, for example, removing a product from a wearer. It is to be appreciated that the lengths, angles, and spacings in transition zones may be different from those in adjacent lines of weakness.

As shown in FIG. 6B for example, the first tear zone 813a of the first frangible pathway 700a may comprise a first initial tear zone 815a extending from the first accessibility opening 802a to a first transition zone 817a. In addition, the first tear zone 813a of the first frangible pathway 700a may comprise a secondary tear zone 819a extending from the first transition zone 817a to the first distal terminus 808a. The first tear zone 813a of the first frangible pathway 700a may also comprise a second initial tear zone 815b extending from the first accessibility opening 802a to a second transition zone 817b. Further, the first tear zone 813a of the first frangible pathway 700a may comprise a second secondary tear zone 819b extending from the second transition zone 817b to the first proximal terminus 810a. The first transition zone 817a may operatively connect the first initial tear zone 815a with the first secondary tear zone 819a to help facilitate the propagation of the tear along the first frangible pathway 700a from first initial tear zone 815a to the first secondary tear zone 819a. With continued reference to FIG. 6B, the first tear zone 813a of the second frangible pathway 700b may comprise a first initial tear zone 815a extending from the second accessibility opening 802b to a first transition zone 817a. In addition, the first tear zone 813a of the second frangible pathway 700b may comprise a secondary tear zone 819a extending from the first transition zone 817a to the second distal terminus 808b. The first tear zone 813a of the second frangible pathway 700b may also comprise a second initial tear zone 815b extending from the second accessibility opening 802b to a second transition zone 817b. Further, the first tear zone 813a of the second frangible pathway 700b may comprise a second secondary tear zone 819b extending from the second transition zone 817b to the second proximal terminus 810b. The second transition zone 817b may operatively connect the second initial tear zone 815b with the second secondary tear zone 819b to help facilitate the propagation of the tear along the second frangible pathway 700b from second initial tear zone 815b to the second secondary tear zone 819b.

As discussed above, the first elastic belt 106 and/or the second belt 108 may be relatively easily torn along the frangible pathway 700, such as when removing the diaper pant 100P from a wearer. As discussed below with reference to FIGS. 6A-6F, the frangible pathway 700 may be configured to allow a caregiver or wearer to initiate and/or completely tear the first belt 106 and/or the second belt 108 with one hand when removing a diaper pant 100P from a wearer. In addition, the first belt 106 may be separable along the first frangible pathway 700a and the second frangible pathway 700b to define a first belt zone 831, a second belt zone 832, and a third belt zone 833 positioned laterally between the first and second belt zones 831, 832.

Referring now to FIGS. 6A and 6B, when removing a diaper pant 100P from a wearer, a user may grab the first belt 106 in the grip region 801 by inserting one or more fingers and/or a thumb through the accessibility opening 802 to grasp a portion of the first 106 and fastener component 707. For example, with reference to FIGS. 6B and 6C, a caregiver may insert a finger or thumb through the first accessibility opening 802a and grasp the first belt 106 and the first fastener component 707a with a first hand. The caregiver's opposing second hand may be used to help stabilize the wearer. For example, the caregiver's opposing second hand may apply a holding or stabilizing force to the wearer at the central region 106c of the first belt 106. The user's first hand may then exert a pulling force Fp on the first grip region 801a of the first belt 106 outward away from the wearer to initiate a tearing of the first belt 106 along the first frangible pathway 700a, such as shown in FIG. 6C.

With continued reference to FIG. 6C, a pulling force Fp (generally represented by an arrow) may be applied to the first grip region 801a in a direction generally toward the first end region 106a of the first belt 106 and/or outward away from the first belt 106 and the wearer. As the force Fp is applied, a first tear line 705a and a second tear line 705b may simultaneously propagate along the first tear zone 813a and the second tear zone 813b, respectively. The first tear line 705a may propagate from the first accessibility opening 802a along the first tear zone 813a of the first frangible pathway 700a in longitudinal and lateral directions partially through and adjacent to the first fastener component 707a and then in a direction D1 that is generally laterally and longitudinally outward from the central region 106c of the first belt 106 and toward the first distal terminus 808a in the first end region 106a of the first belt 106. Simultaneously, the second tear line 705b may propagate from the first accessibility opening 802a in longitudinal and lateral directions partially through and adjacent to the first fastener component 707a along the second tear zone 813b of the first frangible pathway 700a in a direction D2 that is generally laterally outward and longitudinally inward from the central region 106c of the first belt 106 and toward the first proximal terminus 810a in the first end region 106a of the first belt 106.

In some configurations, the first tear line 705a may propagate from the first accessibility opening 802a along the first initial tear zone 815a of the first frangible pathway 700a to the first transition zone 817a. From the first transition zone 817a, the first tear line 705a may then propagate along the first secondary tear zone 819a to the first distal terminus 808a. In addition, the second tear line 705b may propagate from the first accessibility opening 802a along the second initial tear zone 815b of the first frangible pathway 700a to the second transition zone 817b. From the second transition zone 817b, the second tear line 705b may then propagate along the second secondary tear zone 819b to the first proximal terminus 810a. As discussed in more detail below, the first frangible pathway 700a may be configured such that the first tear line 705a and the second tear line 705b may reach first distal terminus 808a and the first proximal terminus 810a, respectively, at the same time or about the same time.

As shown in FIG. 6D, the first belt 106 may be separable along the first frangible pathway 700a to define a first belt zone 831. For example, the first belt zone 831 may be formed once the first tear line 705a propagates through the first distal terminus 808a and the second tear line 705b propagates through to the first proximal terminus 810a, the first belt zone 831 may be formed. As shown in FIG. 6D, a first edge 831a of the first belt zone 831 is formed by tearing the first frangiblc pathway 700a. In addition, a first edge 833a of the third belt zone 833 discussed in more detail below is also formed by tearing the first frangible pathway 700a. The first belt zone 831 may extend from the first edge 831a of the first and second tear lines 705a, 705b to the first side scam 178 or the first longitudinal side edge 111a of the first belt 106. In addition, the first belt zone 831 may include the first fastener component 707a. As discussed below, the first belt zone 831 may include the entirety of or a portion of first fastener component 707a.

With the first belt zone 831 being defined by tearing the first belt 106 along the first frangible pathway 700a, a user may proceed to define the second belt zone 832 by tearing the first belt 106 along the second frangible pathway 700b. Referring now to FIGS. 6D and 6E, a caregiver may insert a finger or thumb through the second accessibility opening 802b and grasp the first belt 106 and the second fastener component 707b with a first hand. The caregiver's opposing second hand may be used to help stabilize the wearer. For example, the caregiver's opposing second hand may apply a holding or stabilizing force to the wearer at the central region 106c of the first belt 106. The user's first hand may then exert a pulling force Fp on the second grip region 801b of the first belt 106 outward away from the wearer to initiate a tearing of the first belt 106 along the second frangible pathway 700b, such as shown in FIG. 6E.

With continued reference to FIG. 6E, a pulling force Fp (generally represented by an arrow) is applied to the second grip region 801b in a direction generally toward the second end region 106b of the first belt 106 and/or outward away from the first belt 106. As the pulling force Fp is applied, a first tear line 705a and a second tear line 705b may simultaneously propagate along the first tear zone 813a and the second tear zone 813b, respectively. The first tear line 705a may propagate from the second accessibility opening 802b along the first tear zone 813a of the second frangible pathway 700b in longitudinal and lateral directions partially through and adjacent to the second fastener component 707b and then in a direction D1 that is generally laterally and longitudinally outward from the central region 106c of the first belt 106 and toward the second distal terminus 808b in the second end region 106b of the first belt 106. Simultaneously, the second tear line 705b may propagate from the second accessibility opening 802b in longitudinal and lateral directions partially through and adjacent to the second fastener component 707b along the second tear zone 813b of the second frangible pathway 700b in a direction D2 that is generally laterally outward and longitudinally inward from the central region 106c of the first belt 106 and toward the second proximal terminus 810b in the second end region 106b of the first belt 106.

In some configurations, the first tear line 705a may propagate from the second accessibility opening 802b along the first initial tear zone 815a of the second frangible pathway 700b to the first transition zone 817a. From the first transition zone 817a, the first tear line 705a may then propagate along the first secondary tear zone 819a to the second distal terminus 808b. In addition, the second tear line 705b may propagate from the second accessibility opening 802b along the second initial tear zone 815b of the second frangible pathway 700b to the second transition zone 817b. From the second transition zone 817b, the second tear line 705b may then propagate along the second secondary tear zone 819b to the second proximal terminus 810b. As discussed in more detail below, the second frangible pathway 700b may be configured such that the first tear line 705a and the second tear line 705b may reach second distal terminus 808b and the second proximal terminus 810b, respectively, at the same time or about the same time.

As shown in FIG. 6F, the first belt 106 may be separable along the second frangible pathway 700b to define a second belt zone 832 and a third belt zone 833. For example, the second belt zone 832 may be formed once the first tear line 705a propagates through the second distal terminus 808b and the second tear line 705b propagates through to the second proximal terminus 810b, the second belt zone 832 may be formed. As shown in FIG. 6F, a first edge 832a of the second belt zone 832 is formed by tearing the second frangible pathway 700b. In addition, a second edge 833b of the third belt zone 833 is also formed by tearing the second frangible pathway 700b. The second belt zone 832 may extend from the first edge 832a of the first and second tear lines 705a, 705b to the second side seam 180 or the second longitudinal side edge 111b of the first belt 106. In addition, the second belt zone 832 may include the second fastener component 707b. The third belt zone 833 may extend laterally between the first edge 833a and the second edge 833b and may remain connected with the chassis 102.

Although the tearing process is described above with reference to FIGS. 6A-6F as tearing the first belt 106 along the first frangible pathway 700a before tearing the first belt along the second frangible pathway 700b, it is to be appreciated that the tearing of first belt 106 along the frangible pathways 700 may occur in various different orders and in different manners. For example, the first belt 106 may be torn along second frangible pathway 700b to define the second belt zone 832 before tearing the first belt 106 along the first frangible pathway 700a to define the first belt zone 831. In another example, the first belt 106 may be torn simultaneously along the first frangible pathway 700a and the second frangible pathway 700b to define the first belt zone 831, the second belt zone 832, and the third belt zone 833.

Once the first belt 106 is torn along the frangible pathways 700 to define the first belt zone 831, the second belt zone 832, and the third belt zone 833, the diaper pant 100P may be removed from a wearer in a manner similar to that of a conventional taped diaper. After being removed from a wearer, the diaper pant 100P may be placed in a disposal configuration, such as discussed above with reference to FIGS. 5A and 5B, by rolling and/or folding the chassis 102 onto itself in a longitudinal direction. The first belt zone 831 and the second belt zone 832 may be used to further wrap the diaper pant 100P onto itself. And the fastener components 707 on the first belt zone 831 and the second belt zone 832 may be connected with another portion of the diaper pant 100P to help maintain the diaper pant 100P in the disposal configuration.

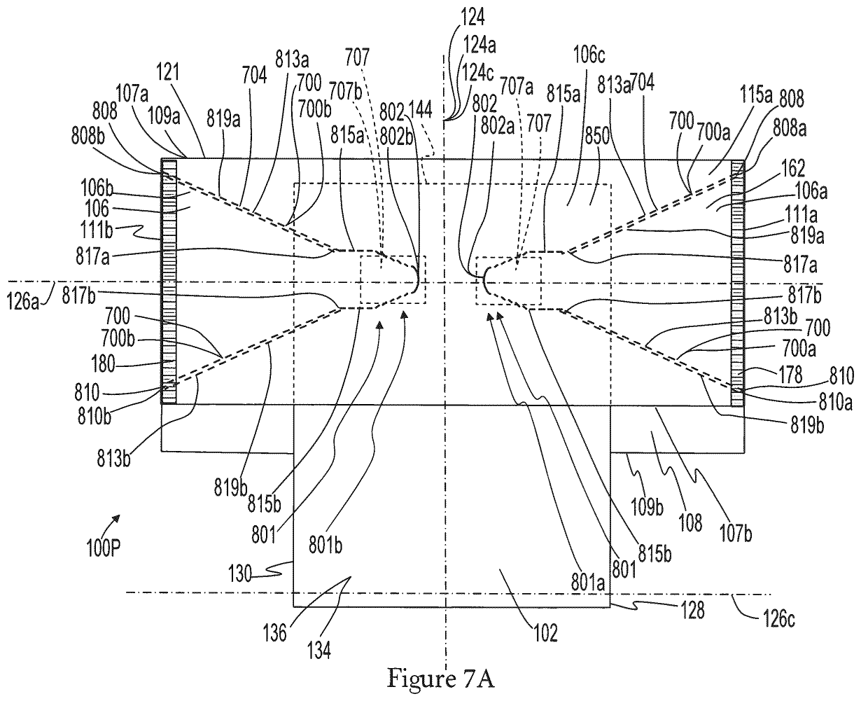

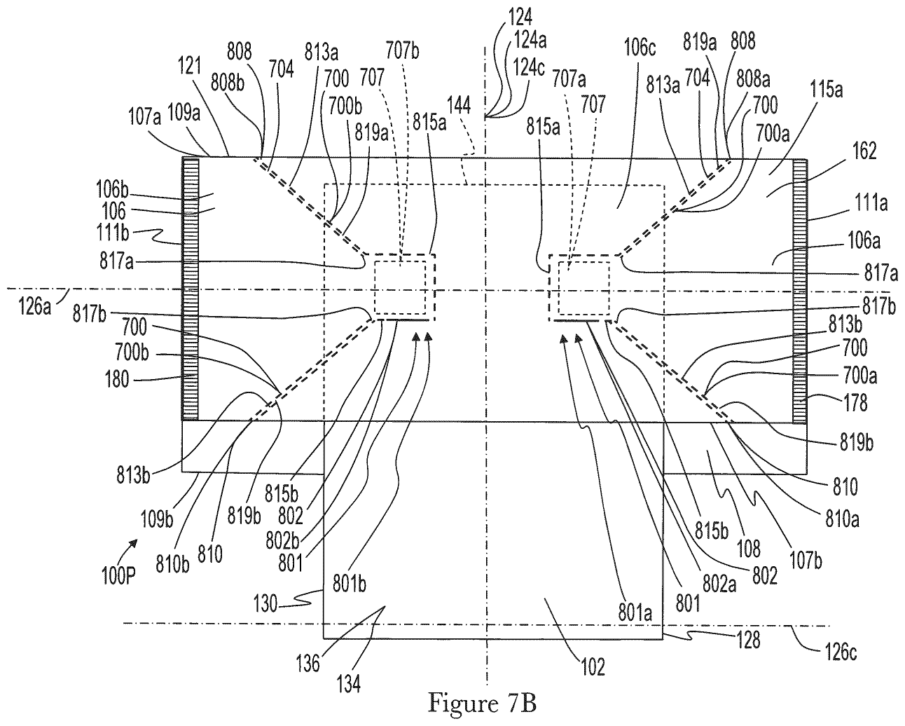

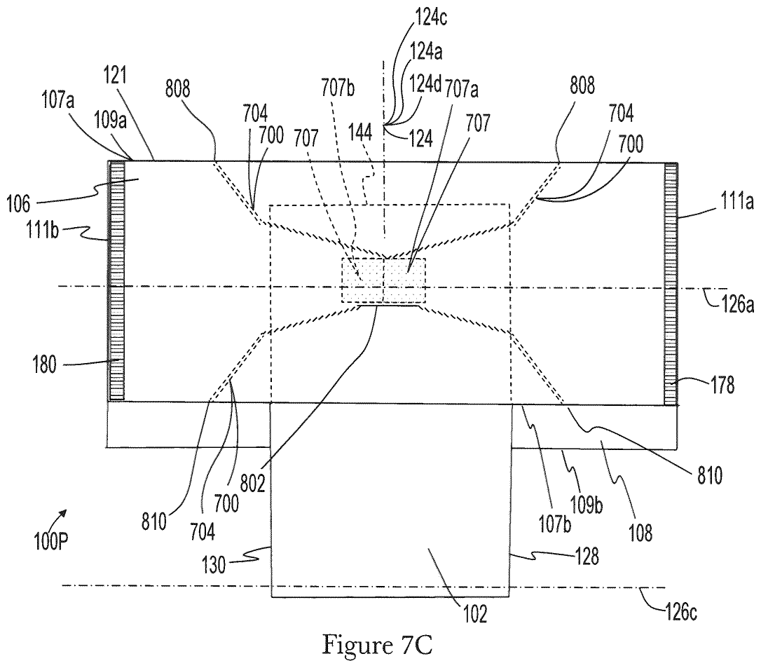

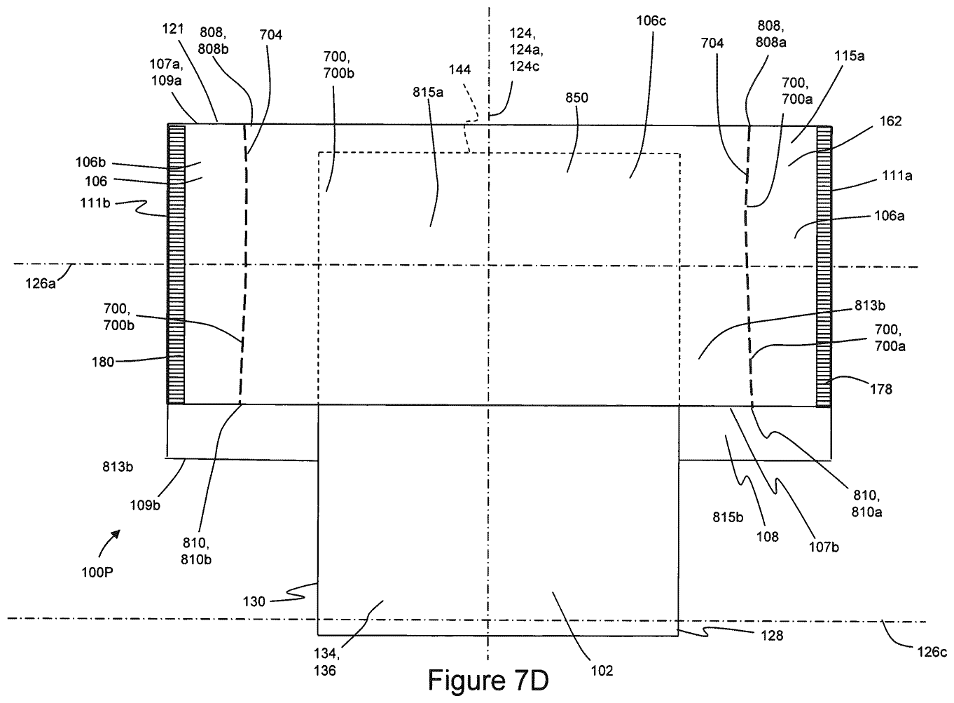

As discussed above, it is to be appreciated that the first distal terminus 808a and the second distal terminus 808b may be located in various longitudinal positions between the outer edge 107a and the inner edge 107b of the first belt 106. And the first proximal terminus 810a and the second proximal terminus 810b may be located in various longitudinal positions between the outer edge 107a and the inner edge 107b of the first belt 106. For example, in some configurations, such as shown in FIG. 7A for example, the first distal terminus 808a and/or the first proximal terminus 810a may be located on the first side seam 178 at positions longitudinally inboard of the outer edge 107a and longitudinally outboard of the inner edge 107b of the first belt 106. Also, as shown in FIG. 7A, the second distal terminus 808b and/or the second proximal terminus 810b may be located on the second side scam 180 at positions longitudinally inboard of the outer edge 107a and longitudinally outboard of the inner edge 107b of the first belt 106. As such, completing the tearing process of the first belt 106 may also require tearing portions of the first and/or second side seams 178, 180.

With reference to various aspects of the Figures described above, it is also to be appreciated that grip regions 801 and accessibility openings 802 may be located in various positions in the first end region 106a, the second end region 106b, and/or the central region 106c of the first belt 106. Grip regions 801 and accessibility openings 802 may be positioned between the first longitudinal side edge 111a, the second longitudinal side edge 111b, the outer edge 107a, and the inner edge 107b of the first belt 106. For example, the first accessibility opening 802a and/or the second accessibility 802b may be entirely laterally positioned between the first longitudinal edge 128 and the second longitudinal edge 130 of the chassis 102. In some configurations, the first accessibility opening 802a may be positioned laterally between the first longitudinal side edge 128 of the chassis 102 and the first longitudinal side edge 111a of the first belt 106 and/or first side scam 178. In some configurations, the second accessibility opening 802b may be positioned laterally between the second longitudinal side edge 130 of the chassis 102 and the second longitudinal side edge 111b of the first belt 106 and/or second side seam 180. In some configurations, the first accessibility opening 802a and/or the second accessibility opening 802b may be positioned longitudinally between the first lateral edge 144 of the chassis 102 and the inner edge 107b of the first belt 106 and/or may be positioned longitudinally between the first lateral edge 144 of the chassis 102 and the outer edge 107a of the first belt 106. In some configurations, the first accessibility opening 802a may extend across the first longitudinal edge 128 and/or the first lateral edge 144 of the chassis 102, and/or the second accessibility opening 802b may extend across the second longitudinal edge 130 and/or the first lateral edge 144 of the chassis 102.

It is also be appreciated that accessibility openings 802 may be located in various positions relative to fastener components 707. For example, in some configurations, the accessibility opening 802 may be positioned longitudinally between the fastener component 707 and the inner edge 107b of the first belt 106, such as shown in FIG. 7B. In some configurations, the accessibility opening 802 may be positioned longitudinally between the fastener component 707 and the outer edge 107a of the first belt 106. In some configurations, the accessibility opening 802 may be positioned laterally inboard of the fastener component 707. It is also to be appreciated that more than one accessibility opening 802 may be located adjacent a fastener component 707. As discussed in more detail below, the accessibility opening 802 also be configured to extend partially or entirely through a fastener component 707 and may divide a fastener component 707 into two or more parts.

As mentioned above, the accessibility opening 802 may comprise slits and/or openings in the first belt 106 and may be curved and/or straight. It is to be appreciated that the accessibility openings 802 may also be oriented in various ways. For example, the accessibility opening 802 may be generally oriented perpendicularly relative to the outer edge 107a and/or the inner edge 107b of the first belt 106. In some configurations, the accessibility opening 802 may be generally oriented parallel relative to the outer edge 107a and/or the inner edge 107b of the first belt 106. In some configurations, the accessibility opening 802 may comprise a slit that extends along a line in a lateral direction to define an angle from about 0 degrees to about 45 degrees with respect to the outer edge 107a and/or the inner edge 107b of the first belt 106, specifically reciting all 1 degree increments within the above-recited range and all ranges formed therein or thereby. In some configurations, the accessibility opening 802 may define a length dimension in the range of about 5 mm to about 50 mm, specifically reciting all 0.1 mm increments within the above-recited range and all ranges formed therein or thereby.

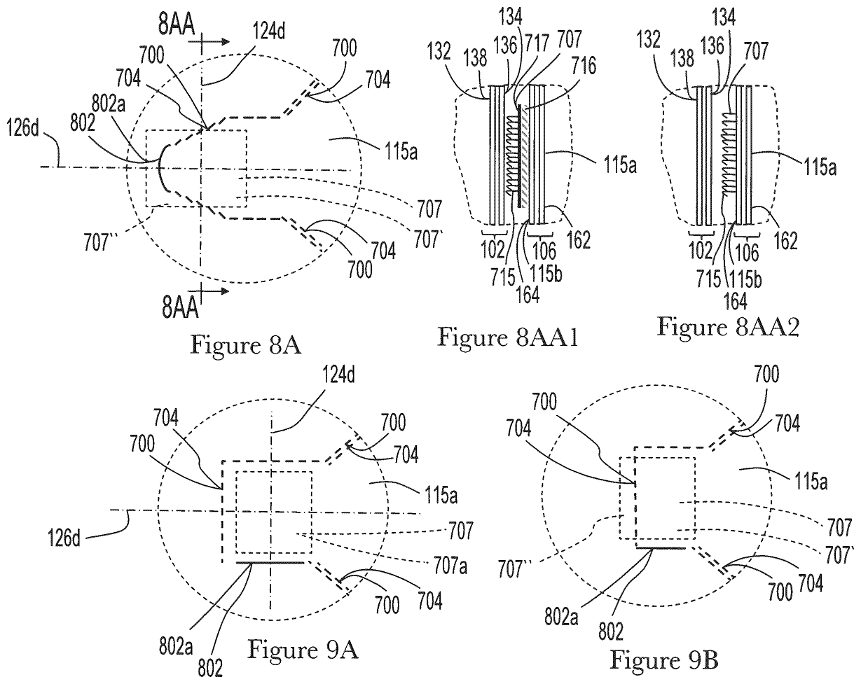

As discussed above, the diaper pant 100P may include one or more fastener components 707 adapted to refastenably connect with at least one other component of the diaper pant 100P in a disposal configuration. It is to be appreciated that the fastener components 707 may be configured in various shapes and sizes, and may be located in various positions relative to other components of the diaper pant 100P. As shown in FIGS. 8A and 9A for example, the fastener components 707 may comprise a lateral centerline 126d oriented substantially parallel to the lateral centerline 126a of the first elastic belt 106 and/or the lateral centerline 126b of the second elastic belt 108 and/or the lateral centerline 126c of the chassis 102. The fastener components 707 may comprise a longitudinal centerline 124d oriented substantially parallel to the longitudinal centerline 124a of the first elastic belt 106 and/or the longitudinal centerline 124b of the second elastic belt 108 and/or the longitudinal centerline 124c of the chassis 102.

As shown in FIG. 8AA1, in some configurations, fastener components 707 may be positioned on and connected with the wearer facing surface 115b of the first elastic belt 106 and/or the second elastic belt 108 in a region where the first elastic belt 106 and/or second elastic belt 108 overlaps the chassis 102. In some configurations, the fastener component 707 may be sandwiched between the second substrate 164 of the first elastic belt 106 or the second elastic belt 108 and the backsheet 136 of the chassis 102. In some configurations, such as shown in FIG. 8AA1, the fastener component 707 comprises hooks 715 protruding from a base 717, and the hooks 715 extend from the first belt 106 toward the backsheet 136. The fastener component 707 may be configured as a separate discrete element that may be connected with the wearer facing surface 115b of the first belt 106 in various ways. For example, as shown in FIG. 8AA1, adhesive 716 may connect the base 717 of the fastener component 707 with wearer facing surface 115b of the first belt 106. It is to be appreciated that the fastener component 707 may be connected with the first belt 106 by mechanical bonding in addition to or instead of adhesive. It is to be appreciated that the base 717 may be configured in various ways. For example, the base 717 may comprise a thermoplastic film. In some configurations, the base 717 may comprise a laminate with various layers bonded together, such as disclosed for example in U.S. Patent Publication No. 2021/0045931 A1. For example, the base 717 may comprise a thermoplastic film layer bonded with a nonwoven layer. It is to be appreciated that such layers may be bonded together in various ways, such as with adhesive, mechanical bonding, and/or extrusion bonding. In some configurations, the fastener component 707 may be integrally formed from materials of the first belt 106, such as shown for example in FIG. 8AA2, or may be integrally formed from materials and attached with the first belt.

As previously mentioned, it is to be appreciated that the fastener components 707 referred to herein may be configured in various ways, such as hooks, loops, and/or adhesive. For example, the fastener component 707 may comprise hook elements or adhesive adapted to refastenably connect with another surface of the diaper pant 100P. In some configurations, the fastener component 707 may comprise loop elements adapted to refastenably connect with hook surface on the diaper pant 100P. The fastener component 707 may be a separate element connected with the clastic belt 106 in various ways, such as mechanical bonding, adhesive bonding, or both. In some configurations, the fastener component 707 may be integrally formed from materials of the elastic belt 106, 108. In some configurations, the fastener component 707 may be printed and/or comprise materials of various different colors such that the fastener component 707 may be visible from outside the diaper pant 100P.

In some configurations, the fastener component 707 may comprise a hook material adapted to refastenably engage with substrates, such as nonwovens for example, on an exterior surface of the diaper pant 100P. For example, the fastener component 707 may comprise a substrate comprising hooks, with the substrate bonded to the clastic belt 106, 108, such as the second substrate 164, which may be in the form of a nonwoven. It is to be appreciated that the substrate may be bonded to the elastic belt 106, 108 in various ways, such as for example, with mechanical bonds, thermal bonds, ultrasonic bonds, and/or adhesive bonds or combinations thereof. In some configurations, hooks may be integrally formed from the second substrate 164, which may be in the form of a nonwoven. The fastener component 707 may comprise one material or a combination of two or more materials arranged in at least partially overlapping configuration. In some configurations, the fastener component 707 may comprise other fastener types as known in the art.

It is to be appreciated that the fastener component 707 may comprise any of a wide variety of shapes, including rectangles or other polygons, circles, ovals, shapes having exterior convexities or concavities or combinations thereof, or one or a plurality of lines or geometric shapes forming an array. It is to be appreciated that the fastener component 707 may comprise various sizes. For example, in some configurations, the fastener component 707 may have a lateral width of between about 5 mm and about 100 mm, specifically reciting all 0.1 mm increments within the above-recited range and all ranges formed therein or thereby. In some configurations, the fastener component 707 may have a longitudinal length of between about 10 mm and about 100 mm, specifically reciting all 0.1 mm increments within the above-recited range and all ranges formed therein or thereby. The fastener component 707 may be aligned parallel the lateral centerline 126a, 126b of the elastic belt 106, 108 or may be oriented at an angle relative the longitudinal centerline 126a, 126 of the clastic belt 106, 108 of between 0 and 90 degrees. The fastener component 707 may comprise an array of two or more spaced-apart fastening elements. The fastener component 707 may have a color that is visible through any layers of the clastic belt 106, 108 on which the fastener component 707 is located. The elastic belt 106, 108 and/or chassis 102 may include printing or other indicia highlighting to a caregiver the location, function, and/or usage of the fastener component 707. The bond, or bond pattern, attaching the fastener component 707 to the clastic belt 106, 108 may be visually or tactilely distinct from the surrounding belt material in order to provide the caregiver a signal or a mechanical grip advantage.

As shown for example in FIG. 6B, a portion of the chassis 102 may overlap the inner wearer facing surface 115b of the first belt 106 to define a chassis overlap region 850. As such, the chassis overlap region 850 may extend laterally between the first longitudinal edge 128 and the second longitudinal edge 130 of the chassis 102 and longitudinally between the first lateral edge 144 of the chassis 102 and the inner edge 107b of the first belt 106. To help prevent contact of the fastener component 707 with a wearer's skin while wearing the diaper pant 100P, the fastener components 707 may be positioned on and connected with the wearer facing surface 115b of the first clastic belt 106 and/or the wearer facing surface 117b of the second clastic belt 108 in the chassis overlap region 850 where the first elastic belt 106 and/or second elastic belt 108 overlaps the chassis 102. For example, the fastener component 707 may be sandwiched between the wearer facing surface 115b of the first belt 106 and the chassis 102. In some configurations, the fastener component 707 may be sandwiched between the second substrate 164 of the first clastic belt 106 or the second elastic belt 108 and the backsheet 136 of the chassis 102. In some configurations, the fastener component 707 may be positioned laterally between the first longitudinal side edge 128 and the second longitudinal side edge 130 of the chassis 102. The fastener component 707 may also be positioned longitudinally between the first lateral edge 144 of the chassis 102 and the inner edge 107b of the first belt 106. As shown in FIG. 9A, the fastener component 707 may be positioned adjacent the frangible pathway 700. In the configuration shown in FIG. 8A, both the frangible pathway 700 and the accessibility opening 802 may extend through the fastener component, effectively dividing the fastener component 707 into a first fastener part 707 and a second fastener part 707″. The accessibility openings 802 shown in FIG. 8A may comprise slits that are generally oriented in a longitudinal direction. In addition, the accessibility opening 802 extends through the fastener component 707 and may be positioned entirely within a perimeter of the fastener component. It is to be appreciated that such slits may be straight and/or curved. The accessibility opening 802, which may be considered part of the frangible pathway 700, may be positioned adjacent the fastener component 707. As such, the frangible pathway 700 may partially surround the fastener component 707. In some configurations, such as shown in FIGS. 8A and 9B, the frangible pathway 700 and/or accessibility opening 802 may extend through the fastener component 700, effectively dividing the fastener component 707 into a first fastener part 707 and a second fastener part 707″. As discussed above, the first fastener part 707 is separated from the second fastener part 707″ as the frangible pathway is torn. When completing the tearing operation, the first belt zone 831 and the second belt zone 832 will include first fastener parts 707, and the third belt zone 833 will include second fastener parts 707″separated from respective first fastener parts 707 during the tearing of frangible pathways 700.

In another configuration shown in FIG. 7C, the diaper pant 100P may comprise one fastener component 707 joined to the wearer facing surface 115b of the first belt 106 in a location overlapping the longitudinal centerline 124c of the chassis 102. The longitudinal centerline 124d of the fastener component 707 may be coincident with, or in proximity of, the longitudinal centerline 124c of the chassis 102. The frangible pathway 700 may divide fastener component 707 into the first fastener component 707a and the second fastener component 707b of substantially similar size and geometry. An accessibility opening 802 may be disposed at, or in proximity of, a longitudinally inboard lateral edge of the fastener component 707. Longitudinally outboard the lateral edges of the fastener component 707, the frangible pathway 700 may extend in longitudinal and lateral directions to the waist edge 121 and inner edge 107b of the first belt 106. A caregiver or wearer may access and grasp the fastener component 707 through the accessibility opening 802 and subsequently separate the frangible pathway 700 into the first and second fastener components 707a, 707b.

In other configurations, such as shown in FIG. 7D, the absorbent article 100P may or may not include fastener components 707 adjacent frangible pathways 700. In some configurations, the frangible pathways 700 may be positioned entirely outside the chassis overlap region 850 and may not overlap the chassis 102. The first frangible pathway 700a may be entirely positioned laterally between the first longitudinal edge 128 of the chassis 102 and the first side seam 178, and the second frangible pathway 700b may be entirely positioned laterally between the second longitudinal edge 130 of the chassis 102 and the second side seam 180.

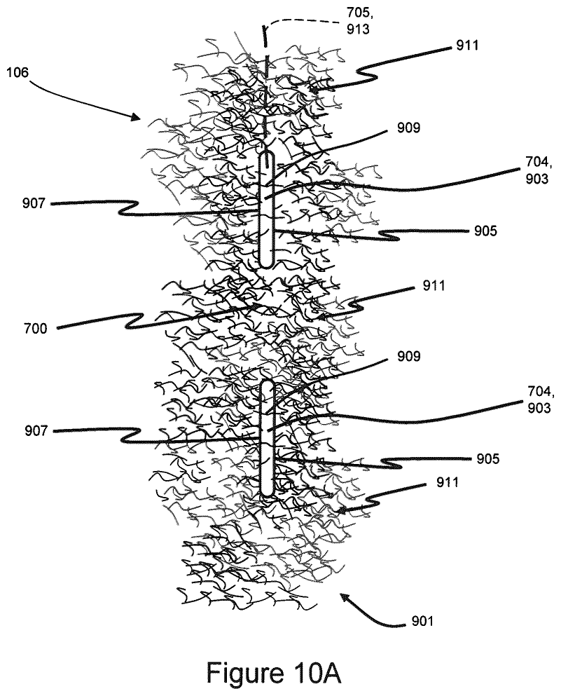

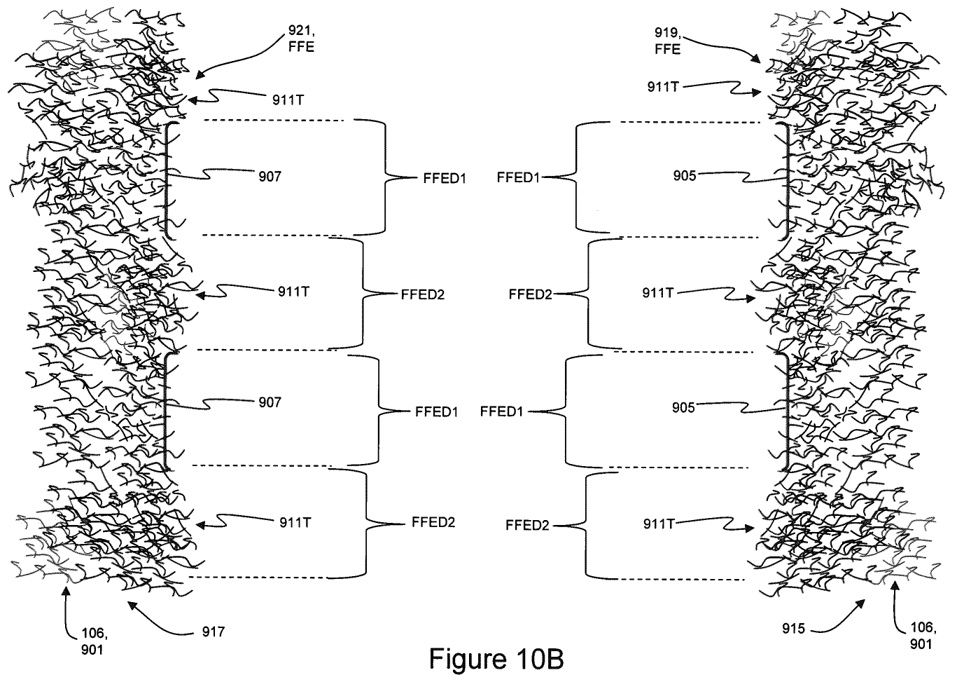

As discussed above, the first belt 106 and/or the second belt 108 may comprise one or more layers of substrates 162, 164, which may comprise fibrous substrates such as nonwovens, for example. In addition, the first belt 106 and/or the second belt 108 may comprise one or more frangible pathways 700 comprising lines of weakness 704. For example, FIG. 10A shows a detailed sketch of lines of weakness 704 in the first belt 106 comprising cut lines 903 that extend through a fibrous substrate 901. It is to be appreciated that the cut lines 903 may be formed with a cutting apparatus that may comprise a knife and anvil for example. In some configurations, the cut lines may be formed by advancing the fibrous substrate 901 through a nip between a knife roll and an anvil roll. As such, pressures between the knife and anvil, referred to herein as cutting pressures, may form cut edges, and in some configurations, such cut edges may comprise materials of one or more layers of belt substrates that are compressed, welded, entangled, and/or fused together. As shown in FIG. 10A, the cut lines 903 may comprise a first cut edge 905 separated from a second cut edge 907.

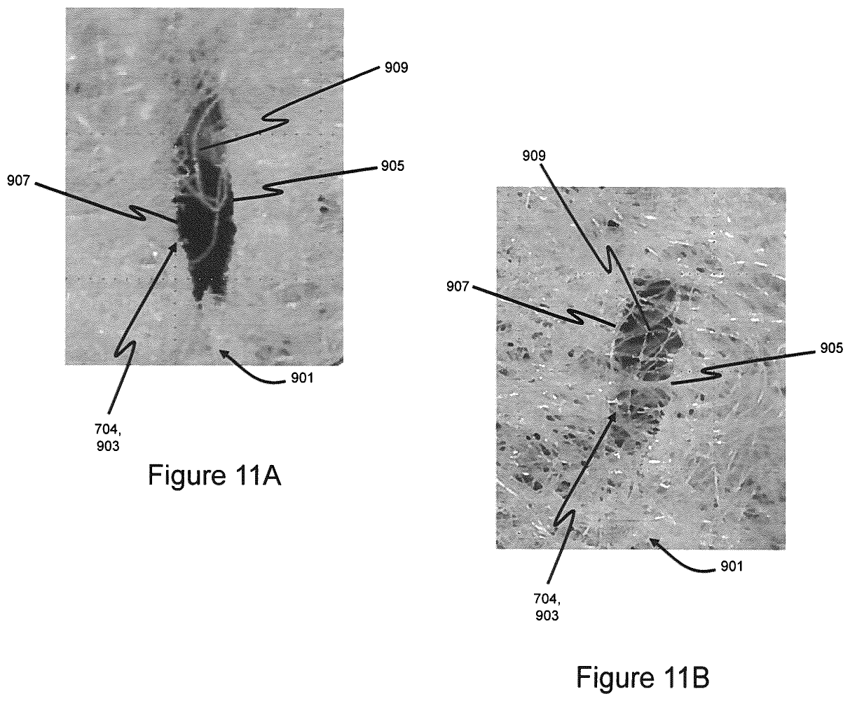

It is to be appreciated that cutting pressures mentioned above may be adjusted to form various configurations of cut lines 903. For example, in some configurations, relatively lower or higher cutting pressures may be utilized when forming the cut lines 903. In turn, relatively lower or higher cutting pressures may create cut lines 903 wherein some uncut or loose fibers of the fibrous substrate 901, referred to herein as remainder fibers 909, exist at the line of weakness 704. Such remainder fibers 909 may not be cut and/or may extend at least partially across the cut line 903 between the first cut edge 905 and the second cut edge 907, such as shown in FIG. 10A. In some configurations, relatively higher cutting pressures may result in relatively fewer numbers of remainder fibers 909 at the line of weakness 704, and relatively lower cutting pressures may result in relatively greater numbers of remainder fibers 909 at the line of weakness 704. For example, FIG. 11A is a microscope photograph of a line of weakness 704 made with a first cutting pressure and showing a relatively few number of remainder fibers 909 extending between first and second cut edges 905, 907 of the cut line 903. And FIG. 11B is a microscope photograph of a line of weakness 704 made with a second cutting pressure that is lower than the first cutting pressure and showing a relatively large number of remainder fibers 909 extending between first and second cut edges 905, 907 of a cut line 903. In some configurations, the remainder fibers 909 may help to visually obscure, cover, and/or hide the existence of the line of weakness 704. In turn, such covered or obscured lines of weakness 704 may help reduce the possibility of drawing unwanted attention from a curious wearer of a diaper pant, such as a toddler. In addition, the remainder fibers 909 may increase the difficulty for a wearer to insert fingers into the lines of weakness 704. In some configurations, the remainder fibers 909 may help provide additional tensile strength to the frangible pathway 700 so as to help reduce the possibility of unintentionally failing during belt and/or article manufacturing operations, while at the same time not significantly increasing the difficulty to tear the belt along the frangible pathway 700.

In some configurations, a Cut Line Occlusion Measurement Method disclosed herein may be used to generate a Cut Line Occlusion value expressed in a percentage to provide an indication of a relative quantity of remainder fibers 909 of a cut line 903. For example, a relatively low cut line occlusion percentage may provide an indication of a relatively low quantity of remainder fibers, whereas a relatively high cut line occlusion percentage may provide an indication of a relatively high quantity of remainder fibers. In some configurations, a cut line 903 may comprise a Cut Line Occlusion of about 15% to about 80%, specifically reciting all 1% increments within the above-recited range and all ranges formed therein or thereby.

As discussed above, the first belt may also include an accessibility opening 802 which may a part of the frangible pathway 700. It is to be appreciated that in some configurations, it may be desirable to form the accessibility opening 802 without remainder fibers 909 extending across the accessibility opening 802 that would otherwise visually obstruct the location and/or increase the difficulty to insert fingers through the accessibility opening 802. As such, accessibility openings 802 may be formed with relatively high cutting pressures to help ensure all fibers are cut and/or significantly reduce the numbers of remainder fibers 909. In some configurations, the belt substrates may be subjected to additional operations to help ensure the accessibility openings 802 are completely opened, such as disclosed in U.S. Patent Application No. 63/610,082, which is incorporated herein by reference.

It is to be appreciated that some belt configurations may include lines of weakness 704 with relatively high quantities of remainder fibers 909 and accessibility openings 802 with relatively few or no remainder fibers 909. It is further to be appreciated that such combinations of lines of weakness 704 and accessibility openings 802 may be formed with the same or substantially the same cutting pressures by utilizing different materials into the areas intended to have different numbers of remainder fibers 909. For example, for a given cutting pressure, quantities of remainder fibers 909 at lines of weakness 704 may be increased by utilizing relatively thin nonwoven fibrous substrates comprising relatively small denier fibers in areas of the belt intended to be cut, while quantities of remainder fibers 909 at accessibility openings 802 may be decreased by adding relative thick polymer patches, such as fastener components for example, to the areas of the belt intended to be cut.

With continued reference to FIG. 10A, the frangible pathway 700 may comprise lines of weakness 704 comprising cut lines 903 extending through the fibrous substrate 901, each line of weakness 903 being separated from another line of weakness 903 by land regions 911, which may also be referred to as “ties.” As discussed above with reference to FIGS. 6A-6F, the first belt 106 is separable along the frangible pathway 700 with tear lines 705a, 705b to define edges of belt zones, such as for example, the first edge 831a of the first belt zone 831, the first edge 832a the second belt zone 832, and/or the first and second edges 833a, 833b of the third belt zone 833.

It is to be appreciated that the above discussed tear lines that may propagate along the frangible pathway 700 and through the cut lines 903 and the land regions 911. A reference line 913 in FIG. 10A is illustrated to show an approximate path along which a tear line 705 may propagate when the tearing the first belt 106 along the frangible pathway 700. FIG. 10B shows a detailed view of the frangible pathway 700 of FIG. 10A after having been separated along the tear line 705 to form a first belt zone 915 and a second belt zone 917. As shown in FIG. 10B, the first belt zone 915 comprises a first edge 919 comprising first cut edges 905 of the divided cut lines 903, which are separated from each other by torn land regions 911T; and the second belt zone 917 comprises a second edge 921 comprising second cut edges 907 of the divided cut lines 903, which are separated from each other by torn land regions 911T.

With continued reference to FIG. 10B, the first edge 919 of the first belt zone 915 and the second edge 921 second belt zone 917 may be defined by free or loose ends of fibers of the fibrous substrate 901, referred to herein as free fiber ends FFE. It is to be appreciated that a measure of the extent of the presence of such free fiber ends FFE may be expressed as free fiber end density FFED, which is the number of free fibers ends FFE of one or more fibrous substrates that are present along a linear length of an edge of a belt zone defined by a tear line 705 that has propagated along a frangible pathway 700. As such, a free fiber end density FFED may expressed in units of “free fiber ends/mm.” As shown in FIG. 10B, the first cut edges 905 and/or the second cut edges 907 may comprise a first free fiber end density FFED1, and the torn land regions 911T may comprise a second free fiber end density FFED2, wherein the second free fiber end density FFED2 is greater than the first free fiber end density FFED1. The first free fiber end density FFED1 and/or the second free fiber end density FFED2 may be greater than zero. It is to be appreciated that the first free fiber end density FFED1 and the second free fiber end density FFED2 may help enhance the perception of relatively soft tear edge and/or may help reduce the occasion in which a user may contact a rough feeling cut edge during the diaper pant removal process. In some configurations, a ratio of the first free fiber end density FFED1 to the second free fiber end density FFED2 is less than 0.5. In some configurations, the first cut edges 905 may comprise a first length and a first free fiber end density FFED1, and the torn land regions 911T may comprise a second length and a second free fiber end density FFED2, and the second free fiber end density FFED2 times the second length of the torn land region 911T is greater than the first free fiber end density FFED1 times the first length of the first cut edge 905. In some configurations, the second cut edges 907 may comprise a first length and a first free fiber end density FFED1, and the torn land regions 911T may comprise a second length and a second free fiber end density FFED2, and the second free fiber end density FFED2 times the second length of the torn land region 911T is greater than the first free fiber end density FFED1 times the first length of the second cut edge 907. It is to be appreciated that the first length of the first cut edge 905 and/or the first length of the second cut edge 907 may be the same as or different from the second length of the torn land regions 911T. In some configurations, the second free fiber end density FFED2 is from about 2 free fiber ends/mm to about 10000 free fiber ends/mm, specifically reciting all 1 free fiber ends/mm increments within the above-recited range and all ranges formed therein or thereby. In some configurations, a torn land region 911T may comprise about 60 free fiber ends/mm.

In some configurations, a Loose Fiber Ends Measurement Method disclosed herein may be used to generate a Loose Fiber Ends Value to provide an indication of a relative quantity of loose fibers along a tear edge. In turn, a Loose Fiber Ends Value may provide an indication a relatively soft tear edge and/or a relatively rough feeling cut edge. A relatively high Loose Fiber Ends Value indicates a relatively high quantity of loose fibers along the tear edge, and a relatively low Loose Fiber Ends Value indicates a relatively low quantity of loose fibers along the tear edge. In some configurations, a first edge 919 and/or a second edge 921 may comprise a Loose Fiber Ends Value of about 35 to about 65, specifically reciting all 1 Value increments within the above-recited range and all ranges formed therein or thereby.

### Loose Fiber Ends Value Measurement Method

Loose Fiber Ends Value measurements are obtained from sample images acquired using a flatbed scanner. The scanner is capable of scanning in reflectance mode at a resolution of 6400 dpi and 8-bit grayscale (a suitable scanner is an Epson Perfection V850 Pro from Epson America Inc., Long Beach CA, or equivalent). The scanner is interfaced with a computer running an image analysis program (a suitable program is ImageJ v. 1.53, or equivalent, National Institute of Health, USA). The acquired sample images are distance calibrated against an acquired image of a ruler certified by NIST. The resulting image is then thresheld, generating a binary image segmenting open regions from sample material regions, and analyzed using the image analysis program. All testing is performed in a conditioned room maintained at about 23±2° C. and about 50±2% relative humidity.

### Sample Preparation:

Test samples are prepared by manually tearing the absorbent article along the frangible pathway in a manner consistent with the product's intended use. The test sample consists of an edge formed by tearing along a frangible pathway. Precondition samples at about 23° C.±2 C.° and about 50%±2% relative humidity for 2 hours prior to testing.

### Image Acquisition:

Place the ruler on the scanner bed, oriented parallel to the sides of the scanner glass, and close the lid. Acquire a calibration image of the ruler in reflectance mode at a resolution of 6400 dpi (approximately 252 pixels per mm) and 8-bit grayscale. Save the calibration image as an uncompressed TIFF format file. Lift the lid and remove the ruler. After obtaining the calibration image, all samples are scanned under the same conditions and measured based on the same calibration file. Next, place the torn edge of the sample onto the center of the scanner bed, lying flat against the scanner's glass surface. Cover the sample with a matte black tile, allowing the tile to press the loose fibers along the torn edge against the glass, and close the lid. Acquire a scanned image of the torn edge of the sample and save the image as an uncompressed TIFF format file.

### Loose Fiber Ends Value Measurement:

Open the calibration image (containing the ruler) file in the image analysis program. Perform a linear distance calibration using the imaged ruler. This distance calibration scale will be applied to all subsequent sample images prior to analysis. Open the sample image in the image analysis program. Set the distance scale. Next, apply an Unsharp Mask filter to increase the contrast along the loose fiber and material edges where the dark background meets the lighter material edges. The unsharp masking technique sharpens edges within an image by subtracting a blurred (unsharp) version of the image from itself. Finally, threshold the image at a gray level value that separates the visibly identifiable boundaries between the open regions from the loose fibers and material along the torn edge of the sample to generate a binary image where the loose fibers and material portion of the sample are in the foreground.

Using the image analysis program, identify and outline the exterior boundaries of the loose fibers and material foreground objects in the binary image, as well as boundaries of holes inside these objects. Measure and record the total length of all the identified boundaries by summing up the number of pixels in the identified boundary outlines and convert this length to millimeters using the resolution of the scanned image. Exclude any boundary pixels from the outer perimeter of the image itself, background noise, or separated fibers that are not an attached part of the scanned sample. Divide this total boundary length by the width, in mm, of the scanned portion of the sample to obtain a normalized total boundary length to sample width ratio and report this Loose Fiber Ends Value to the nearest whole number. A higher Loose Fiber Ends Value indicates a greater number of loose fibers along the torn edge of the sample.

### Cut Line Occlusion Measurement Method

Cut Line Occlusion measurements are obtained from sample images acquired using a flatbed scanner. The scanner is capable of scanning in reflectance mode at a resolution of 6400 dpi and 8-bit grayscale (a suitable scanner is an Epson Perfection V850 Pro from Epson America Inc., Long Beach CA, or equivalent). The scanner is interfaced with a computer running an image analysis program (a suitable program is ImageJ v. 1.53, or equivalent, National Institute of Health, USA). The acquired sample images are distance calibrated against an acquired image of a ruler certified by NIST. The resulting image is then thresheld, separating open regions from sample material regions, and analyzed using the image analysis program. All testing is performed in a conditioned room maintained at about 23±2° C. and about 50±2% relative humidity.

### Sample Preparation:

Test samples are prepared by first excising a region of the absorbent article containing cut lines along a frangible pathway from the absorbent article. This excised sample of the absorbent article containing cut lines along a frangible pathway is then fully extended thereby opening the cut lines revealing any uncut fibers occluding the opening. The sample is then secured in this fully extended state to a matte black tile enhancing contrast and visibility of any uncut fibers occluding the cut opening against the black background. Precondition samples at about 23° C.±2 C.° and about 50%±2% relative humidity for 2 hours prior to extending the belt sample for testing.

### Image Acquisition:

Place the ruler on the scanner bed, oriented parallel to the sides of the scanner glass, and close the lid. Acquire a calibration image of the ruler in reflectance mode at a resolution of 6400 dpi (approximately 252 pixels per mm) and 8-bit grayscale. Save the calibration image as an uncompressed TIFF format file. Lift the lid and remove the ruler. After obtaining the calibration image, test samples are scanned under the same conditions and measured based on the same calibration file. Next, place the tile with the attached extended belt sample onto the center of the scanner bed, with the sample lying flat against the scanner's glass surface backed with the black tile and close the lid. Acquire a scanned image of an individual cut line and save the image as an uncompressed TIFF format file.

### Cut Line Occlusion Measurement:

Open the calibration image (containing the ruler) file in the image analysis program. Perform a linear distance calibration using the imaged ruler. This distance calibration scale will be applied to all subsequent sample images prior to analysis. Open the sample image in the image analysis program. Set the distance scale. Next, apply an Unsharp Mask to increase the contrast along the cut line opening and uncut fiber edges where the dark background meets the lighter material edges. The unsharp masking technique sharpens edges within an image by subtracting a blurred (unsharp) version of the image from itself. Then manually trace the visibly identifiable boundary of the cut line opening. Measure and record the area of the cut line opening. Finally, threshold the image at a gray level value that separates the visibly identifiable boundaries between the open regions from the uncut fibers and material along the cut line opening boundary to generate a binary image where the uncut fibers and material portion of the sample are in the foreground and the open regions in the background.

Using the image analysis program, analyze each of the discrete background regions within the identified boundary of the cut line opening. Measure and record all the individual background region areas. Sum all the individual areas and divide this total by the recorded area of the entire cut line opening and subtract this value from one to calculate the portion of occluded area. Record and report this value as the Cut Line Occlusion as a percentage to the nearest whole percent.

### Average Decitex (Average-Dtex)

The Average Decitex Method is used to calculate the Average-Dtex on a length-weighted basis for elastic fibers present in an entire article, or in a specimen of interest extracted from an article. The decitex value is the mass in grams of a fiber present in 10,000 meters of that material in the relaxed state. The decitex value of elastic fibers or elastic laminates containing elastic fibers is often reported by manufacturers as part of a specification for an elastic fiber or an elastic laminate including elastic fibers. The Average-Dtex is to be calculated from these specifications if available. Alternatively, if these specified values are not known, the decitex value of an individual elastic fiber is measured by determining the cross-sectional area of a fiber in a relaxed state via a suitable microscopy technique such as scanning electron microscopy (SEM), determining the composition of the fiber via Fourier Transform Infrared (FT-IR) spectroscopy, and then using a literature value for density of the composition to calculate the mass in grams of the fiber present in 10,000 meters of the fiber. The manufacturer-provided or experimentally measured decitex values for the individual elastic fibers removed from an entire article, or specimen extracted from an article, are used in the expression below in which the length-weighted average of decitex value among elastic fibers present is determined.

The lengths of elastic fibers present in an article or specimen extracted from an article is calculated from overall dimensions of and the elastic fiber pre-strain ratio associated with components of the article with these or the specimen, respectively, if known. Alternatively, dimensions and/or elastic fiber pre-strain ratios are not known, an absorbent article or specimen extracted from an absorbent article is disassembled and all elastic fibers are removed. This disassembly can be done, for example, with gentle heating to soften adhesives, with a cryogenic spray (e.g., Quick-Freeze, Miller-Stephenson Company, Danbury, CT), or with an appropriate solvent that will remove adhesive but not swell, alter, or destroy elastic fibers. The length of each elastic fiber in its relaxed state is measured and recorded in millimeters (mm) to the nearest mm.

### Calculation of Average-Dtex

For each of the individual elastic fibers fᵢ of relaxed length Lᵢ and fiber decitex value dᵢ (obtained either from the manufacturer's specifications or measured experimentally) present in an absorbent article, or specimen extracted from an absorbent article, the Average-Dtex for that absorbent article or specimen extracted from an absorbent article is defined as:

$$Average - Dtex = \frac{{\sum }_{i = 1}^{n} ( {L}_{i} \times {d}_{i} )}{{\sum }_{i = 1}^{n} {L}_{i}}$$

where n is the total number of elastic fibers present in an absorbent article or specimen extracted from an absorbent article. The Average-Dtex is reported to the nearest integer value of decitex (grams per 10 000 m).

If the decitex value of any individual fiber is not known from specifications, it is experimentally determined as described below, and the resulting fiber decitex value(s) are used in the above equation to determine Average-Dtex.

### Experimental Determination of Decitex Value for a Fiber

For each of the elastic fibers removed from an absorbent article or specimen extracted from an absorbent article according to the procedure described above, the length of each elastic fiber Lₖ in its relaxed state is measured and recorded in millimeters (mm) to the nearest mm. Each elastic fiber is analyzed via FT-IR spectroscopy to determine its composition, and its density βₖ is determined from available literature values. Finally, each fiber is analyzed via SEM. The fiber is cut in three approximately equal locations perpendicularly along its length with a sharp blade to create a clean cross-section for SEM analysis. Three fiber segments with these cross sections exposed are mounted on an SEM sample holder in a relaxed state, sputter coated with gold, introduced into an SEM for analysis, and imaged at a resolution sufficient to clearly elucidate fiber cross sections. Fiber cross sections are oriented as perpendicular as possible to the detector to minimize any oblique distortion in the measured cross sections. Fiber cross sections may vary in shape, and some fibers may consist of a plurality of individual filaments. Regardless, the area of each of the three fiber cross sections is determined (for example, using diameters for round fibers, major and minor axes for elliptical fibers, and image analysis for more complicated shapes), and the average of the three areas αₖ for the elastic fiber, in units of micrometers squared (μm²), is recorded to the nearest 0.1 μm². The decitex dₖ of the kth elastic fiber measured is calculated by:

$${d}_{k} = 10 \; 000 m \times {a}_{k} \times {ρ}_{k} \times 1 {0}^{- 6}$$

where dₖ is in units of grams (per calculated 10,000 meter length), αₖ is in units of μm², and ρₖ is in units of grams per cubic centimeter (g/cm³). For any elastic fiber analyzed, the experimentally determined Lₖ and dₖ values are subsequently used in the expression above for Average-Dtex.

### Average-Strand-Spacing

Using a ruler calibrated against a certified NIST ruler and accurate to 0.5 mm, measure the distance between the two distal strands within a section to the nearest 0.5 mm, and then divide by the number of strands in that section-1

Average-Strand-Spacing=d/(n−1) where n>1
report to the nearest 0.1 mm.

### Average-Pre-Strain

The Average-Pre-Strain of a specimen are measured on a constant rate of extension tensile tester (a suitable instrument is the MTS Insight using Testworks 4.0 Software, as available from MTS Systems Corp., Eden Prairie, MN) using a load cell for which the forces measured are within 1% to 90% of the limit of the cell. Articles are conditioned at 23° C.±2 C.° and 50%±2% relative humidity for 2 hours prior to analysis and then tested under the same environmental conditions.

Program the tensile tester to perform an elongation to break after an initial gage length adjustment. First raise the cross head at 10 mm/min up to a force of 0.05N. Set the current gage to the adjusted gage length. Raise the crosshead at a rate of 100 mm/min until the specimen breaks (force drops 20% after maximum peak force). Return the cross head to its original position. Force and extension data is acquired at a rate of 100 Hz throughout the experiment.

Set the nominal gage length to 40 mm using a calibrated caliper block and zero the crosshead. Insert the specimen into the upper grip such that the middle of the test strip is positioned 20 mm below the grip. The specimen may be folded perpendicular to the pull axis, and placed in the grip to achieve this position. After the grip is closed the excess material can be trimmed. Insert the specimen into the lower grips and close. Once again, the strip can be folded, and then trimmed after the grip is closed. Zero the load cell. The specimen should have a minimal slack but less than 0.05 N of force on the load cell. Start the test program.

From the data construct a Force (N) verses Extension (mm). The Average-Pre-Strain is calculated from the bend in the curve corresponding to the extension at which the nonwovens in the clastic are engaged. Plot two lines, corresponding to the region of the curve before the bend (primarily the elastics), and the region after the bend (primarily the nonwovens). Read the extension at which these two lines intersect, and calculate the % Pre-Strain from the extension and the corrected gage length. Record as % Pre-strain 0.1%. Calculate the arithmetic mean of three replicate samples for each elastomeric laminate and Average-Pre-Strain to the nearest 0.1%.

### Combinations

A1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first free fiber end density, and wherein the torn land regions comprise a second free fiber end density; and wherein the second free fiber end density is greater than the first free fiber end density.

A2. The absorbent article of paragraph A1, wherein the second free fiber end density is from about 2 free fiber ends/mm to about 10000 free fiber ends/mm.

A3. The absorbent article of either paragraph A1 or A2, wherein the second free fiber end density is greater than zero.

A4. The absorbent article of any of paragraphs A1 to A3, wherein the first free fiber end density is greater than zero.

A5. The absorbent article of any of paragraphs A1 to A4, wherein a portion of the chassis overlaps the inner wearer facing surface of the first belt to define a chassis overlap region.

A6. The absorbent article of paragraph A5, wherein the frangible pathway extends between a proximal terminus and a distal terminus.

A7. The absorbent article of paragraph A6, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

A8. The absorbent article of any of paragraphs A1 to A6, wherein the frangible pathway further comprises an accessibility opening in the first belt.

A9. The absorbent article of paragraph A8, the frangible pathway comprises a first tear zone extending between the accessibility opening and one of the distal terminus and the proximal terminus.

A10. The absorbent article of paragraph A8, wherein the frangible pathway comprises a second tear zone extending between the accessibility opening and another one of the distal terminus and the proximal terminus.

A11. The absorbent article of paragraph A8, wherein the accessibility opening is positioned in the overlap region.

A12. The absorbent article of any of paragraphs A1 to A11, further comprising a fastener component positioned between the inner wearer facing surface of the first belt and the backsheet.

A13. The absorbent article of paragraph A12, wherein the accessibility opening extends through the fastener component.

A14. The absorbent article of paragraph A5, wherein the frangible pathway does not extend across the overlap region.

A15. The absorbent article of any of paragraphs A1 to A14, wherein first belt comprises at least two layers of fibrous substrates.

A16. The absorbent article of paragraph A15, wherein the first belt further comprises elastic strands sandwiched between the at least two layers of fibrous substrates.

A17. The absorbent article of paragraph A15, wherein a plurality of the clastic strands are severed at the lines of weakness.

A18. The absorbent article of any of paragraphs A1 to A17, wherein the fibrous substrate comprises a nonwoven.

A19. The absorbent article of any of paragraphs A1 to A18, wherein the first edge comprises a Loose Fiber Ends Value of about 35 to about 65.

A20. The absorbent article of any of paragraphs A1 to A19, wherein the cut line comprises a Cut Line Occlusion of about 15% to about 80%.

B1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first free fiber end density, and wherein the torn land regions comprise a second free fiber end density; and wherein a ratio of the first free fiber end density to the second free fiber end density is less than 0.5.

B2. The absorbent article of paragraph B1, wherein the second free fiber end density is from about 2 free fiber ends/mm to about 10000 free fiber ends/mm.

B3. The absorbent article of either paragraph B1 or B2, wherein the second free fiber end density is greater than zero.

B4. The absorbent article of any of paragraphs B to B3, wherein the first free fiber end density is greater than zero.

B5. The absorbent article of any of paragraphs B to B4, wherein a portion of the chassis overlaps the inner wearer facing surface of the first belt to define a chassis overlap region.

B6. The absorbent article of paragraph B5, wherein the frangible pathway extends between a proximal terminus and a distal terminus.

B7. The absorbent article of paragraph B6, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

B8. The absorbent article of any of paragraphs B1 to B6, wherein the frangible pathway further comprises an accessibility opening in the first belt.

B9. The absorbent article of paragraph B8, the frangible pathway comprises a first tear zone extending between the accessibility opening and one of the distal terminus and the proximal terminus.

B10. The absorbent article of paragraph B8, wherein the frangible pathway comprises a second tear zone extending between the accessibility opening and another one of the distal terminus and the proximal terminus.

B11. The absorbent article of paragraph B8, wherein the accessibility opening is positioned in the overlap region.

B12. The absorbent article of any of paragraphs B1 to B11, further comprising a fastener component positioned between the inner wearer facing surface of the first belt and the backsheet.

B13. The absorbent article of paragraph B12, wherein the accessibility opening extends through the fastener component.

B14. The absorbent article of paragraph B5, wherein the frangible pathway does not extend across the overlap region.

B15. The absorbent article of any of paragraphs B1 to B14, wherein first belt comprises at least two layers of fibrous substrates.

B16. The absorbent article of paragraph B15, wherein the first belt further comprises elastic strands sandwiched between the at least two layers of fibrous substrates.

B17. The absorbent article of paragraph B15, wherein a plurality of the clastic strands are severed at the lines of weakness.

B18. The absorbent article of any of paragraphs B1 to B17, wherein the fibrous substrate comprises a nonwoven.

B19. The absorbent article of any of paragraphs B1 to B18, wherein the first edge comprises a Loose Fiber Ends Value of about 35 to about 65.

B20. The absorbent article of any of paragraphs B1 to B19, wherein the cut line comprises a Cut Line Occlusion of about 15% to about 80%.

C1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first length and a first free fiber end density, and wherein the torn land regions comprise a second length and a second free fiber end density; and wherein the second free fiber end density times the second length of the torn land region is greater than the first free fiber end density times the first length of the first cut edge.

C2. The absorbent article of paragraph C1, wherein the second free fiber end density is from about 2 free fiber ends/mm to about 10000 free fiber ends/mm.

C3. The absorbent article of either paragraph C1 or C2, wherein the second free fiber end density is greater than zero.

C4. The absorbent article of any of paragraphs C1 to C3, wherein the first free fiber end density is greater than zero.

C5. The absorbent article of any of paragraphs C1 to C4, wherein a portion of the chassis overlaps the inner wearer facing surface of the first belt to define a chassis overlap region.

C6. The absorbent article of paragraph C5, wherein the frangible pathway extends between a proximal terminus and a distal terminus.

C7. The absorbent article of paragraph C6, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

C8. The absorbent article of any of paragraphs C1 to C6, wherein the frangible pathway further comprises an accessibility opening in the first belt.

C9. The absorbent article of paragraph C8, the frangible pathway comprises a first tear zone extending between the accessibility opening and one of the distal terminus and the proximal terminus.

C10. The absorbent article of paragraph C8, wherein the frangible pathway comprises a second tear zone extending between the accessibility opening and another one of the distal terminus and the proximal terminus.

C11. The absorbent article of paragraph C8, wherein the accessibility opening is positioned in the overlap region.

C12. The absorbent article of any of paragraphs C1 to C11, further comprising a fastener component positioned between the inner wearer facing surface of the first belt and the backsheet.

C13. The absorbent article of paragraph C12, wherein the accessibility opening extends through the fastener component.

C14. The absorbent article of paragraph C5, wherein the frangible pathway does not extend across the overlap region.

C15. The absorbent article of any of paragraphs C1 to C14, wherein first belt comprises at least two layers of fibrous substrates.

C16. The absorbent article of paragraph C15, wherein the first belt further comprises elastic strands sandwiched between the at least two layers of fibrous substrates.

C17. The absorbent article of paragraph C15, wherein a plurality of the elastic strands are severed at the lines of weakness.

C18. The absorbent article of any of paragraphs C1 to C17, wherein the fibrous substrate comprises a nonwoven.

C19. The absorbent article of any of paragraphs C1 to C18, wherein the first length and the second length are the same or different.

C20. The absorbent article of any of paragraphs C1 to C19, wherein the first edge comprises a Loose Fiber Ends Value of about 35 to about 65.

C21. The absorbent article of any of paragraphs C1 to C20, wherein the cut line comprises a Cut Line Occlusion of about 15% to about 80%.

D1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions, and wherein the first cut edges comprise a first free fiber end density, and wherein the torn land regions comprise a second free fiber end density; and wherein the second free fiber end density is from about 2 free fiber ends/mm to about 10000 free fiber ends/mm.

D2. The absorbent article of paragraph D1, wherein the first free fiber end density is greater than zero.

D3. The absorbent article of paragraph D1, wherein a portion of the chassis overlaps the inner wearer facing surface of the first belt to define a chassis overlap region.

D4. The absorbent article of paragraph D3, wherein the frangible pathway extends between a proximal terminus and a distal terminus.

D5. The absorbent article of paragraph D4, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

D6. The absorbent article of paragraph D4, wherein the frangible pathway further comprises an accessibility opening in the first belt.

D7. The absorbent article of paragraph D6, the frangible pathway comprises a first tear zone extending between the accessibility opening and one of the distal terminus and the proximal terminus.

D8. The absorbent article of paragraph D6, wherein the frangible pathway comprises a second tear zone extending between the accessibility opening and another one of the distal terminus and the proximal terminus.

D9. The absorbent article of paragraph D6, wherein the accessibility opening is positioned in the overlap region.

D10. The absorbent article of paragraph D9, further comprising a fastener component positioned between the inner wearer facing surface of the first belt and the backsheet.

D11. The absorbent article of paragraph D10, wherein the accessibility opening extends through the fastener component.

D12. The absorbent article of paragraph D3, wherein the frangible pathway does not extend across the overlap region.

D13. The absorbent article of any of paragraphs D1 to D12, wherein first belt comprises at least two layers of fibrous substrates.

D14. The absorbent article of paragraph D13, wherein the first belt further comprises elastic strands sandwiched between the at least two layers of fibrous substrates.

D15. The absorbent article of paragraph D13, wherein a plurality of the elastic strands are severed at the lines of weakness.

D16. The absorbent article of any of paragraphs D1 to D15, wherein the fibrous substrate comprises a nonwoven.

D17. The absorbent article of any of paragraphs D1 to D16, wherein the first length and the second length are the same or different.

D18. The absorbent article of any of paragraphs D1 to D17, wherein the first edge comprises a Loose Fiber Ends Value of about 35 to about 65.

D19. The absorbent article of any of paragraphs D1 to D18, wherein the cut line comprises a Cut Line Occlusion of about 15% to about 80%.

E1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; and a plurality of fibers of the fibrous substrate extending at least partially across the cut line between the first cut edge and the second cut edge; and wherein the first belt is separable along the frangible pathway with a tear line propagating through the cut lines and the land regions to define a first edge of a first belt zone and a second edge of a second belt zone.

F1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions; and wherein the first edge comprises a Loose Fiber Ends Value of about 35 to about 65.

F2. The absorbent article of paragraph F1, wherein a portion of the chassis overlaps the inner wearer facing surface of the first belt to define a chassis overlap region.

F3. The absorbent article of paragraph F2, wherein the frangible pathway extends between a proximal terminus and a distal terminus.

F4. The absorbent article of paragraph F3, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

F5. The absorbent article of any of paragraphs F1 to F3, wherein the frangible pathway further comprises an accessibility opening in the first belt.

F6. The absorbent article of paragraph F5, the frangible pathway comprises a first tear zone extending between the accessibility opening and one of the distal terminus and the proximal terminus.

F7. The absorbent article of paragraph F5, wherein the frangible pathway comprises a second tear zone extending between the accessibility opening and another one of the distal terminus and the proximal terminus.

F8. The absorbent article of paragraph F5, wherein the accessibility opening is positioned in the overlap region.

F9. The absorbent article of any of paragraphs F1 to F8, further comprising a fastener component positioned between the inner wearer facing surface of the first belt and the backsheet.

F10. The absorbent article of paragraph F9, wherein the accessibility opening extends through the fastener component.

F11. The absorbent article of paragraph F2, wherein the frangible pathway does not extend across the overlap region.

F12. The absorbent article of any of paragraphs F1 to F11, wherein first belt comprises at least two layers of fibrous substrates.

F13. The absorbent article of paragraph F12, wherein the first belt further comprises elastic strands sandwiched between the at least two layers of fibrous substrates.

F14. The absorbent article of paragraph F12, wherein a plurality of the elastic strands are severed at the lines of weakness.

F15. The absorbent article of any of paragraphs F1 to F14, wherein the fibrous substrate comprises a nonwoven.

G1. An absorbent article comprising: a first belt comprising an inner wearer facing surface and an outer garment facing surface, the first belt further comprising at least one layer of a fibrous substrate; a second belt, wherein laterally opposing end portions of the second belt are connected with laterally opposing end portions of the first belt at a first side seam and a second side seam to form a waist opening; a chassis comprising a topsheet, a backsheet, and an absorbent core positioned between the topsheet and the backsheet, and wherein longitudinally opposing end regions of the chassis are connected with the first belt and the second belt; a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate; a plurality of fibers of the fibrous substrate extending at least partially across the cut line between the first cut edge and the second cut edge; wherein the first belt is separable along the frangible pathway with a tear line propagating through the cut lines and the land regions to define a first edge of a first belt zone and a second edge of a second belt zone; wherein the first edge of the first belt zone comprises first cut edges separated from each other by torn land regions; and wherein the cut line comprises a Cut Line Occlusion of about 15% to about 80%.

G2. The absorbent article of paragraph G1, wherein a portion of the chassis overlaps the inner wearer facing surface of the first belt to define a chassis overlap region.

G3. The absorbent article of paragraph G2, wherein the frangible pathway extends between a proximal terminus and a distal terminus.

G4. The absorbent article of paragraph G3, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

G5. The absorbent article of any of paragraphs G1 to G3, wherein the frangible pathway further comprises an accessibility opening in the first belt.

G6. The absorbent article of paragraph G5, the frangible pathway comprises a first tear zone extending between the accessibility opening and one of the distal terminus and the proximal terminus.

G7. The absorbent article of paragraph G5, wherein the frangible pathway comprises a second tear zone extending between the accessibility opening and another one of the distal terminus and the proximal terminus.

G8. The absorbent article of paragraph G5, wherein the accessibility opening is positioned in the overlap region.

G9. The absorbent article of any of paragraphs G1 to G8, further comprising a fastener component positioned between the inner wearer facing surface of the first belt and the backsheet.

G10. The absorbent article of paragraph G9, wherein the accessibility opening extends through the fastener component.

G11. The absorbent article of paragraph G2, wherein the frangible pathway does not extend across the overlap region.

G12. The absorbent article of any of paragraphs G1 to G11, wherein first belt comprises at least two layers of fibrous substrates.

G13. The absorbent article of paragraph G12, wherein the first belt further comprises elastic strands sandwiched between the at least two layers of fibrous substrates.

G14. The absorbent article of paragraph G12, wherein a plurality of the elastic strands are severed at the lines of weakness.

G15. The absorbent article of any of paragraphs G1 to G14, wherein the fibrous substrate comprises a nonwoven.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a first belt comprising an inner wearer facing surface and an outer garment facing surface, the first belt further comprising at least one layer of a fibrous substrate;
a second belt, wherein laterally opposing end portions of the second belt are connected with laterally opposing end portions of the first belt at a first side seam and a second side seam to form a waist opening;
a chassis comprising a topsheet, a backsheet, and an absorbent core positioned between the topsheet and the backsheet, and wherein longitudinally opposing end regions of the chassis are connected with the first belt and the second belt;
a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate;
a plurality of fibers of the fibrous substrate extending at least partially across the cut line between the first cut edge and the second cut edge;
wherein the first belt is separable along the frangible pathway with a tear line propagating through the cut lines and the land regions to define a first edge of a first belt zone and a second edge of a second belt zone;
wherein the first edge of the first belt zone comprises first cut edges separated from each other by torn land regions, and wherein the first cut edges comprise a first free fiber end density, and wherein the torn land regions comprise a second free fiber end density; and
wherein the second free fiber end density is greater than the first free fiber end density.

2. The absorbent article of claim 1, wherein the second free fiber end density is from about 2 free fiber ends/mm to about 10000 free fiber ends/mm.

3. The absorbent article of claim 1, wherein the second free fiber end density is greater than zero.

4. The absorbent article of claim 1, wherein the first free fiber end density is greater than zero.

5. The absorbent article of claim 1, wherein a portion of the chassis overlaps the inner wearer facing surface of the first belt to define a chassis overlap region.

6. The absorbent article of claim 5, wherein the frangible pathway extends between a proximal terminus and a distal terminus.

7. The absorbent article of claim 6, wherein the first belt further comprises a laterally extending inner edge and a laterally extending outer edge, the outer edge positioned longitudinally outward of the inner edge, wherein the proximal terminus is positioned on the inner edge and a distal terminus is positioned on the outer edge.

8. The absorbent article of claim 6, wherein the frangible pathway further comprises an accessibility opening in the first belt.

9. The absorbent article of claim 8, the frangible pathway comprises a first tear zone extending between the accessibility opening and one of the distal terminus and the proximal terminus.

10. The absorbent article of claim 8, wherein the frangible pathway comprises a second tear zone extending between the accessibility opening and another one of the distal terminus and the proximal terminus.

11. The absorbent article of claim 8, wherein the accessibility opening is positioned in the overlap region.

12. The absorbent article of claim 11, further comprising a fastener component positioned between the inner wearer facing surface of the first belt and the backsheet.

13. The absorbent article of claim 12, wherein the accessibility opening extends through the fastener component.

14. The absorbent article of claim 5, wherein the frangible pathway does not extend across the overlap region.

15. The absorbent article of claim 1, wherein first belt comprises at least two layers of fibrous substrates.

16. The absorbent article of claim 15, wherein the first belt further comprises elastic strands sandwiched between the at least two layers of fibrous substrates.

17. The absorbent article of claim 15, wherein a plurality of the elastic strands are severed at the lines of weakness.

18. The absorbent article of claim 1, wherein the fibrous substrate comprises a nonwoven.

19. The absorbent article of claim 1, wherein the first edge comprises a Loose Fiber Ends Value of about 35 to about 65.

20. The absorbent article of claim 1, wherein the cut line comprises a Cut Line Occlusion of about 15% to about 80%.

21. An absorbent article comprising:
a first belt comprising an inner wearer facing surface and an outer garment facing surface, the first belt further comprising at least one layer of a fibrous substrate;
a second belt, wherein laterally opposing end portions of the second belt are connected with laterally opposing end portions of the first belt at a first side seam and a second side seam to form a waist opening;
a chassis comprising a topsheet, a backsheet, and an absorbent core positioned between the topsheet and the backsheet, and wherein longitudinally opposing end regions of the chassis are connected with the first belt and the second belt;
a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate;
a plurality of fibers of the fibrous substrate extending at least partially across the cut line between the first cut edge and the second cut edge;
wherein the first belt is separable along the frangible pathway with a tear line propagating through the cut lines and the land regions to define a first edge of a first belt zone and a second edge of a second belt zone;
wherein the first edge of the first belt zone comprises first cut edges separated from each other by torn land regions; and
wherein the first edge comprises a Loose Fiber Ends Value of about 35 to about 65.

22. The absorbent article of claim 21, wherein a portion of the chassis overlaps the inner wearer facing surface of the first belt to define a chassis overlap region.

23. The absorbent article of claim 22, wherein the frangible pathway further comprises an accessibility opening in the first belt.

24. The absorbent article of claim 23, wherein the accessibility opening is positioned in the overlap region.

25. The absorbent article of claim 24, further comprising a fastener component positioned between the inner wearer facing surface of the first belt and the backsheet, and wherein the accessibility opening extends through the fastener component.

26. An absorbent article comprising:
a first belt comprising an inner wearer facing surface and an outer garment facing surface, the first belt further comprising at least one layer of a fibrous substrate;
a second belt, wherein laterally opposing end portions of the second belt are connected with laterally opposing end portions of the first belt at a first side seam and a second side seam to form a waist opening;
a chassis comprising a topsheet, a backsheet, and an absorbent core positioned between the topsheet and the backsheet, and wherein longitudinally opposing end regions of the chassis are connected with the first belt and the second belt;
a frangible pathway in the first belt, wherein the frangible pathway comprises lines of weakness comprising cut lines extending through the fibrous substrate, each line of weakness being separated from another line of weakness other by land regions, wherein each cut line comprises a first cut edge separated from a second cut edge and wherein each land region comprises an uncut region of the fibrous substrate;
a plurality of fibers of the fibrous substrate extending at least partially across the cut line between the first cut edge and the second cut edge;
wherein the first belt is separable along the frangible pathway with a tear line propagating through the cut lines and the land regions to define a first edge of a first belt zone and a second edge of a second belt zone;
wherein the first edge of the first belt zone comprises first cut edges separated from each other by torn land regions; and
wherein the cut line comprises a Cut Line Occlusion of about 15% to about 80%.

27. The absorbent article of claim 26, wherein a portion of the chassis overlaps the inner wearer facing surface of the first belt to define a chassis overlap region.

28. The absorbent article of claim 27, wherein the frangible pathway further comprises an accessibility opening in the first belt.

29. The absorbent article of claim 28, wherein the accessibility opening is positioned in the overlap region.

30. The absorbent article of claim 29, further comprising a fastener component positioned between the inner wearer facing surface of the first belt and the backsheet, and wherein the accessibility opening extends through the fastener component.
